(19) United States
(12) Patent Application Publication
(10) Pub. No.: US 2019/0019632 A1
(43) Pub. Date: Jan. 17, 2019

(54) ULTRATHIN GRAPHENE-PROTEIN SUPERCAPACITORS

(71) Applicant: University of Connecticut, Farmington, CT (US)

(72) Inventors: James F. Rusling, Farmington, CT (US); Islam M. Mosa, Farmington, CT (US); Challa V. Kumar, Ashford, CT (US); Ajith Pattammattel, Farmington, CT (US)

(21) Appl. No.: 16/035,351

(22) Filed: Jul. 13, 2018

Related U.S. Application Data
(60) Provisional application No. 62/532,198, filed on Jul. 13, 2017.

(51) Int. Cl. H01G 11/58; H01G 11/66; H01G 11/84; A61B 5/00; A61N 1/378

(52) U.S. Cl. CPC H01G 11/58; H01G 11/66; H02J 7/025; A61B 5/00; A61N 1/3785; H01G 11/84

(57) ABSTRACT

Supercapacitors having bilayers of reduced biophilized graphene oxide and a protein nanospacer and methods of producing the same. Also disclosed are implantable biomedical devices including a supercapacitor having bilayers of reduced biophilized graphene oxide and a protein nanospacer.

### CROSS-REFERENCE TO RELATED APPLICATIONS

This application is a non-provisional of and claims the benefit of U.S. Provisional Patent Application No. 62/532,198, filed on Jul. 13, 2017, the contents of which are incorporated herein by reference.

### STATEMENT REGARDING FEDERALLY SPONSORED RESEARCH OR DEVELOPMENT

This invention was made with government support under grant numbers ES003154, EB014586, and EB016707, awarded by the National Institutes of Health and grant number DMR1441879, awarded by the National Science Foundation. The government has certain rights in the invention.

### BACKGROUND OF THE INVENTION

All implantable devices are currently powered by batteries. However, batteries suffer from several limitations: they are bulky, which limits device miniaturization; they have limited lifespan; they are made with toxic materials and electrolytes that can be dangerous if leakage occurs; and they have slow discharge rate of signal in pacemakers, for example.

### SUMMARY OF THE INVENTION

In one embodiment, the invention provides a supercapacitor including reduced biophilized graphene oxide and a protein nanospacer.

In another embodiment, the invention provides a method of producing a supercapacitor including reduced biophilized graphene oxide and a protein nanospacer.

In yet another embodiment, the invention provides an implantable biomedical device, which includes a supercapacitor including reduced biophilized graphene oxide and a protein nanospacer.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustrating an application for a brGO/Mb supercapacitor (central panel) as a micro-power source for biomedical implants.

FIG. 2 illustrates a method of electrode fabrication. (a) Preparation of cationized bovine serum albumin (cBSA) by coupling COOH groups of BSA with tetraethylenepentamine. (b) Adsorption of cBSA onto GO sheets. (c) Gold current collector with a monolayer of mercaptopropionic acid and a layer of polycation PDDA adsorbed on top. (d) Adsorption of bGO. (e) Adsorption of Mb. (f) Film from LbL assembly of three bGO layers alternated with two Mb layers (bGO/Mb). (g) Electrochemically reduced film (brGO/Mb).

FIG. 3 shows characterization results of a graphene-protein hybrid electrode. (a-d) Tapping mode AFM images of LbL assembled film on a mica disc: (a) PDDA, (b) PDDA-bGO, (c) PDDA-bGO-Mb, and (d) PDDA-bGO-Mb-bGO. (e) Change in mass/area and nominal thickness of the film as a function of number of layers assembled, as measured by quartz crystal microbalance (QCM). (f) Raman spectra of films before and after electrochemical reduction, showing an increase in ratio of intensities of D to G bands from 0.94 to 1.14 upon reduction. (g) Myoglobin binding to GO (red curve) and bGO (blue curve) in 10 mM acetate buffer at pH 5.5 as a function of increasing concentrations of myoglobin. (h) Circular dichroism spectra, suggesting extensive denaturation of Mb upon binding to either GO or bGO. (i) Areal capacitance of three bilayers of brGO/protein in correlation to the number of sites available for protonation/deprotonation normalized by the molar mass of proteins. Cyt-C, Mb, Hb, GOx, and Cat stand for cytochrome-C, myoglobin, hemoglobin, glucose oxidase, and catalase, respectively. The highest areal capacitance among these was observed with brGO/Mb film.

FIG. 4 shows electrochemical behavior of a brGO/Mb supercapacitor. (a) Cyclic voltammetry (CV) at 300 mV/s of non-reduced film (bGO/Mb) and of electrochemically reduced film (brGO/Mb) for 3 and 70 seconds. (b) Optimization of film capacitance with number of bilayers, showing highest capacitance for 3 bilayers. (c) Photograph of a flexible brGO/Mb supercapacitor. (d) Cyclic voltammetry (CV) at 100 mV/s of brGO/Mb film in different electrolytes. (e) Change of volumetric and gravimetric capacitance with scan rate for a brGO/Mb electrode in biofluids and acid electrolyte. (f) Cyclic stability of a brGO/Mb bEC in calf serum; inset shows LED powered by three brGO/Mb bECs connected in series with calf serum saturated separators. (g) Volumetric stack capacitance of a brGO/Mb supercapacitor at different current densities as compared to commercial electrical double layer capacitors (EDLC). (h) Energy and power densities of a brGO/Mb supercapacitor in a cell culture medium as compared to commercial EDLC, 300 μF/3V aluminum electrolytic capacitor, 12 μAh/3.3 V Li thin film battery, and 500 μAh/5 V Li thin film battery.

FIG. 5 shows performance of a brGO/Mb supercapacitor in cell culture medium. (a) Photograph of EC device dipped in a 6-well plate containing MEF cells in DMEM, and connected to an electrochemical workstation via copper tape. (b) Scheme showing movement of ions available in cell culture medium during charging process. (c) Charge/discharge cycles at different current densities. (d) CV curves at different scan rates. (e)-(f) Optical microscopy images for MEF cells stained with trypan blue subjected to 0 and 5000 charge discharge cycles, respectively. Both (e) and (f) show live cells appeared with a very small number of dead cells (stained blue-green, black arrows show examples). The absence of differences suggests insignificant cell death due to brGO/Mb supercapacitor operation in the cell culture. (g)-(h) Optical microscopy images for COS-7 cells stained with trypan blue. (g) Control cells with zero charge/discharge cycles. (h) Cells subjected to long-term cycling test of the polydimethylsiloxane (PDMS)-packaged bEC for 96 hours (90,000 cycles), showing no difference in cell death as compared to control cells.

FIG. 6 shows electrochemical impedance spectroscopy (EIS) for a brGO/Mb supercapacitor in biofluids and acid electrolyte. (a) Nyquist plot with high frequency region magnified (inset), showing nearly identical impedance behavior for the device in different electrolytes. The brGO/Mb supercapacitor exhibited low series resistance in acid (4Ω) compared with biofluids (8 to 10Ω). (b) Change of phase angle with frequency; time constant (τ) calculated from frequency (f) at an angle of −45° according to the following relation: τ (seconds) =1/f (Hz) at −45°. (c) Three brGO/Mb supercapacitors connected in series with calf serum saturated separators powering an LED.

FIG. 7 shows charge discharge curves for different proteins LbL assembled with three brGO layers, followed by electrochemical reduction. The brGO/Mb film showed the highest capacitance among other proteins. Mb: myoglobin, Cyt-C: cytochrome C, Hb: hemoglobin, GOx: glucose oxidase, Cat: catalase.

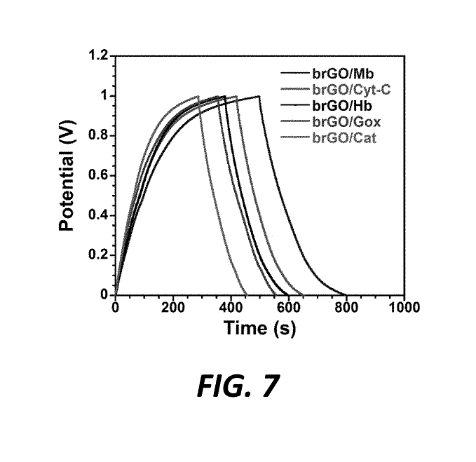

FIG. 8 shows cyclic voltammograms of films of brGO assembled with different proteins on a pyrolytic graphite electrode in a three-electrode system at a scan rate of 1000 mV/s in 1.0 M Na₂SO₄ as a neutral electrolyte. Cationic proteins were prepared in buffers at pHs lower than the isoelectric point of each tested protein. LbL assembly of three bGO layers with two protein layers were carried out, followed by electrochemical reduction. The brGO/Mb film showed the highest capacitance among other proteins. Mb: myoglobin, Hb: haemoglobin, Cyt-C: cytochrome C, GOx: glucose oxidase, Cat: catalase.

FIG. 9 shows SEM images of a continuous brGO/Mb film adsorbed onto a striated, micro-digitated gold current collector. (a) SEM image showing an edge adsorbed brGO/Mb film on the left half versus a bare gold CD surface on the right half. (b) Magnification of the film edge.

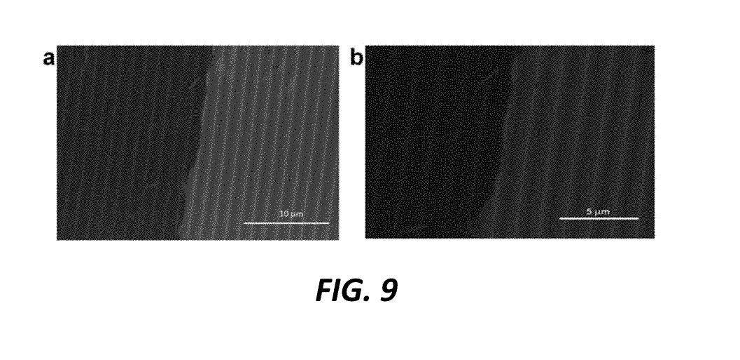

FIG. 10 shows focused ion beam-scanning electron microscope (FIB-SEM) images measuring brGO/Mb film thickness on a gold CD underlayer. Prior to cutting with a focused ion beam, a selected zone was coated with a platinum layer to protect the sample from being damaged by the ion beam. The figure shows a side view of the brGO/Mb film on a gold under layer with platinum coating on the top; the image shows a uniform gold underlayer (42±8 nm), with a uniform brGO/Mb film (26±5 nm) on the gold.

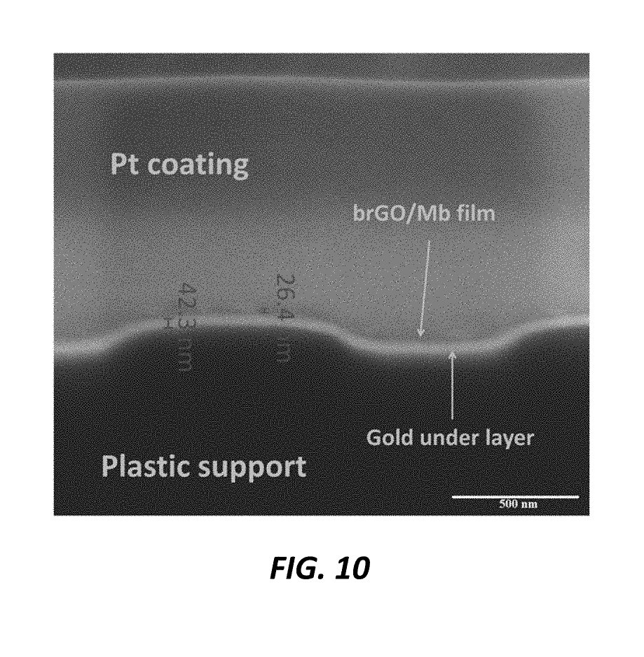

FIG. 11 shows a cross-section of a thick film of brGO/Mb prepared by drop casting on a glass slide to visualize the coating of proteins on the GO layers.

FIG. 12 shows XRD powder patterns of as prepared GO in comparison to LbL assembled bGO/Mb film, showing a shift of the XRD peak to smaller diffraction angle, indicating the role of myoglobin in spacing the GO sheets.

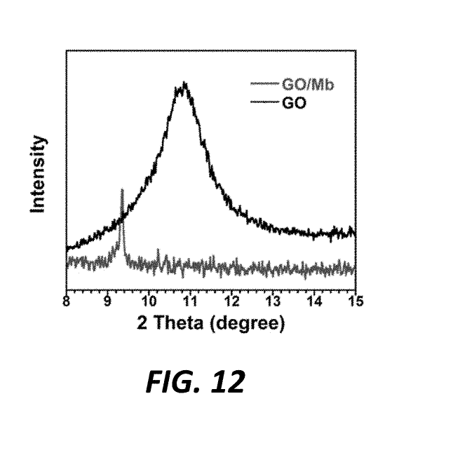

FIG. 13 shows Raman spectra for graphene oxide (GO) before and after binding to different proteins. The spectra show a higher intensity 2D band for GO bound to myoglobin compared to all other proteins or GO alone.

FIG. 14 shows a comparison of capacitive behavior of composites of previously cationized graphene oxide (bGO) and native graphene oxide (GO), both assembled in three bilayers with Mb and electrochemically reduced using the same procedure. Also 5 μg/cm² of GO was loaded to electrodes, electrochemically reduced, and tested for capacitive behavior for comparison. CVs were recorded at 1000 mV/s in 1.0 M Na₂SO₄ as a neutral electrolyte in a three-electrode system, using pyrolytic graphite as the working electrode. The figure demonstrates the superior performance of the bGO containing film.

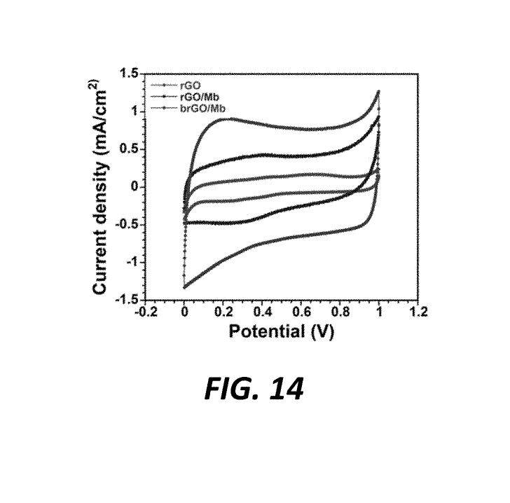

FIG. 15 shows electrochemical performance of brGO/Mb electrodes vs. brGO electrodes (without Mb) in a three electrode cell, with SCE as a reference electrode. (a) Cyclic voltammograms at 0.005 V/s in 0.1 M LiClO₄. (b) Charge/discharge cycle at 2 A/g in 0.1 M LiClO₄.

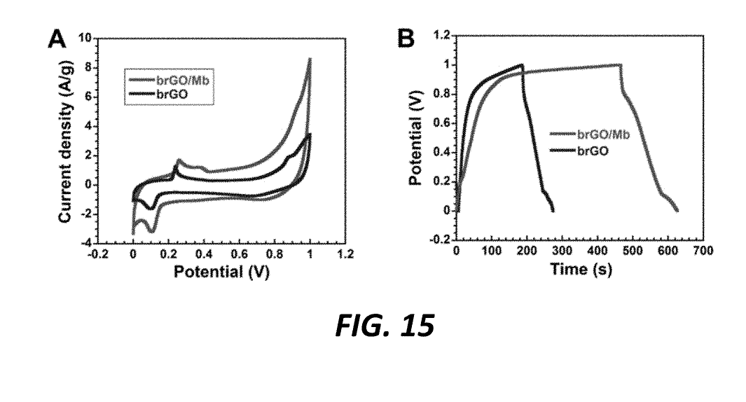

FIG. 16 illustrates the electrochemical reduction, and its effects on capacitance. (a) Repetitive cyclic voltammograms for electrochemical reduction of a bGO/Mb film on pyrolytic graphite. CV indicates disappearance of the reduction peak at −0.95 after the first cycle, confirming an irreversible reduction of bGO to brGO. (b) Cyclic voltammogram for a bGO/Mb film before reduction, showing characteristic myoglobin peaks. (c) Comparison of CV before (red) and after (blue) reduction, causing a dramatic increase in capacitance and a significant decrease of Mb peaks. All CVs were taken at 100 mV/s, in 10 mM acetate buffer, pH 5.5, containing 0.5 M potassium bromide. For more efficient reduction, amperometric reduction at a more negative potential (−1.2 V versus SCE) was used for reducing bGO/Mb film.

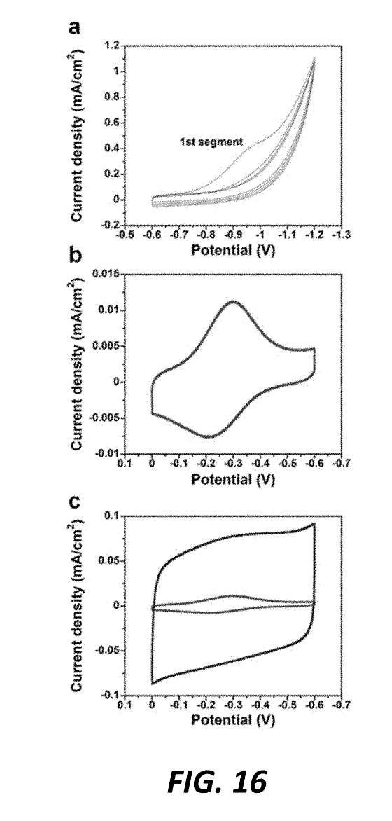

FIG. 17 shows electrochemical performance of Mb electrodes. (a) Cyclic voltammogram at a scan rate of 0.025 V/s in 0.1 M Na₂SO₄. (b) Charge/discharge cycle of Mb electrode at 6 A/g.

FIG. 18 shows the effect of temperature on electrical conductivity of a brGO/Mb electrode.

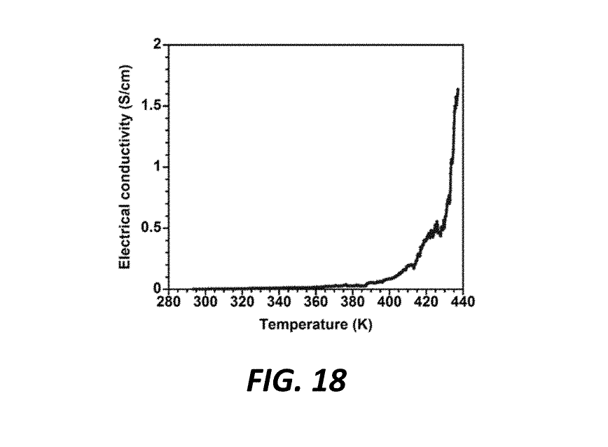

FIG. 19 shows cyclic voltammograms, showing the effects of increasing the number of bilayers of brGO/Mb on current density. LbL assembly of the films was carried out on pyrolytic graphite electrodes and tested in a three-electrode cell in 1.0 M Na₂SO₄ electrolyte at 1000 mV/s. Three bilayers of brGO/Mb showed the highest current density with no further enhancement from adding more layers.

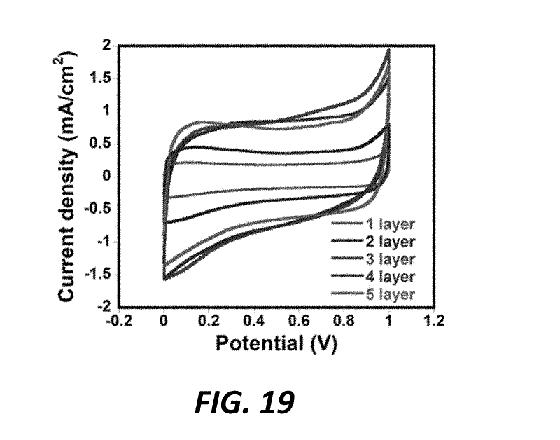

FIG. 20 shows charge discharge curves for the different layers of a brGO/Mb film in calf serum at a current density of 0.05 mA/cm². All layers were assembled on a Au-PDDA current collector.

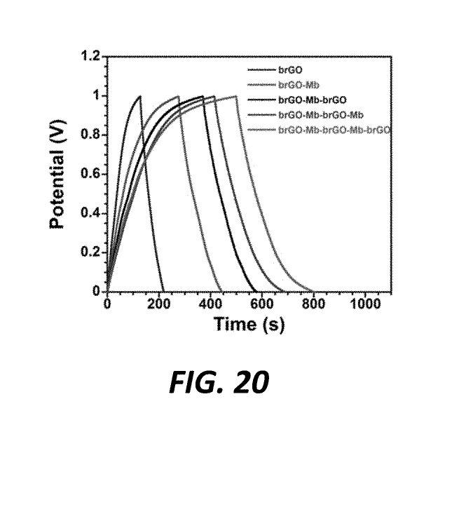

FIG. 21 shows charge/discharge curves for a brGO/Mb electrode in comparison to support materials in calf serum at 0.05 mA/cm².

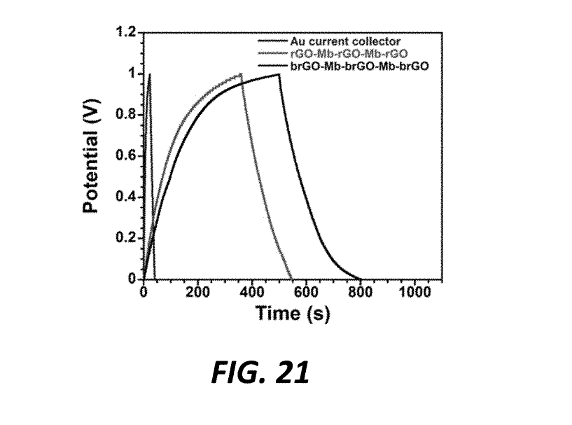

FIG. 22 shows FIB-SEM images measuring PVA separator thickness on gold CD underlayer. Prior to cutting with a focused ion beam, a selected zone was coated with a platinum layer to protect the sample from being damaged by the ion beam. The figure shows a side view of one of the porous PVA separators with an average thickness of 992 nm. The figure shows the plastic support of the CD, the thin gold layer used as the current collector, the PVA porous membrane, the platinum protective coating, and the damaged unprotected PVA membrane.

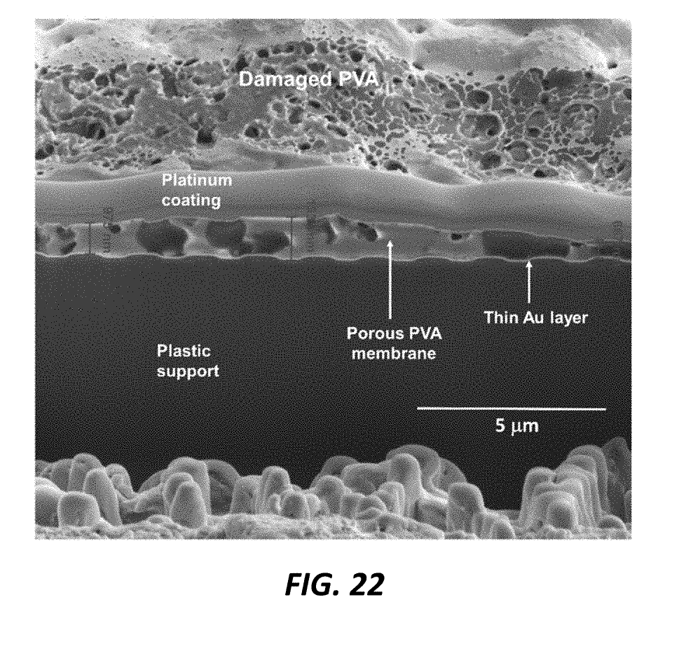

FIG. 23 shows electrochemical behavior of a brGO/Mb supercapacitor in 1.0 M H₂SO₄. (a) Galvanostatic charge/discharge curves at different current densities (2.5 to 25 A/g). (b) Cyclic voltammograms at different scan rates (25 to 2000 mV/s), showing nearly rectangular CV curves up to 500 mV/s.

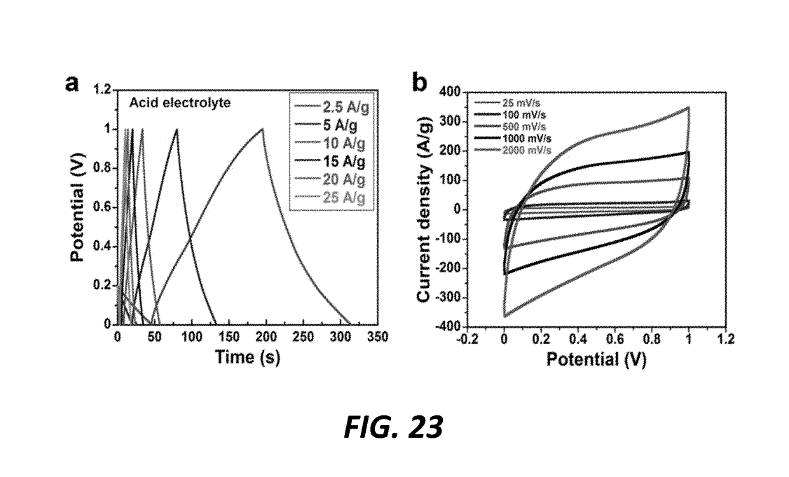

FIG. 24 shows: (a) Change of volumetric and gravimetric capacitance with scan rate for a brGO/Mb electrode in biofluids and acid electrolyte; and (b) Cyclic stability of the brGO/Mb EC in calf serum compared with acid. The inset shows the charge-discharge curves in acid medium at a current density of 25 A/g.

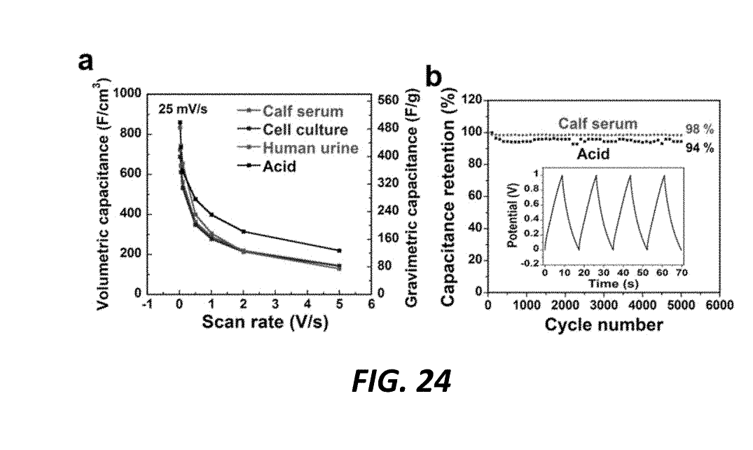

FIG. 25 shows electrochemical behavior of a brGO/Mb supercapacitor in calf serum. (a) Galvanostatic charge/discharge curves at different current densities (2.5 to 25 A/g). (b) Cyclic voltammograms at different scan rates (25 to 2000 mV/s).

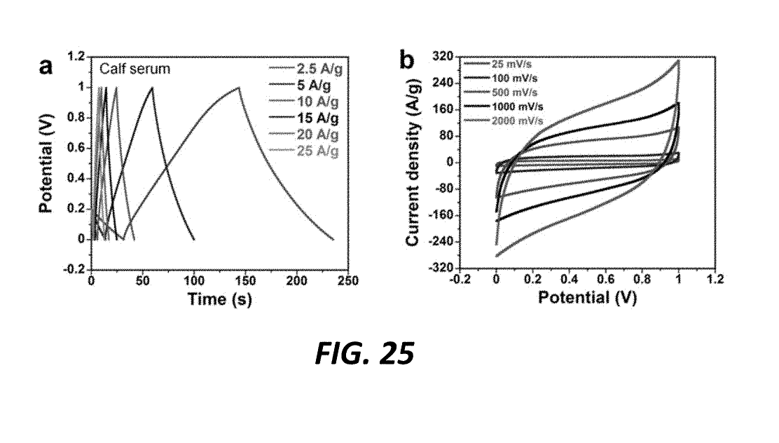

FIG. 26 shows electrochemical behavior of a brGO/Mb supercapacitor in human urine. (a) Galvanostatic charge/discharge curves at different current densities (2.5 to 25 A/g). (b) Cyclic voltammograms at different scan rates (25 to 2000 mV/s).

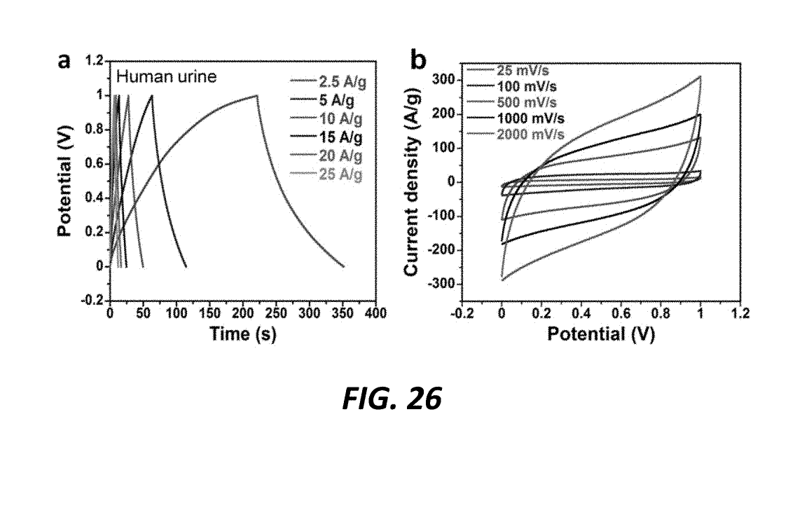

FIG. 27 shows a powder X-ray diffraction (PXRD) pattern of brGO/Mb. (B) Barrett-Joyner-Halenda (BJH) desorption pore size distribution.

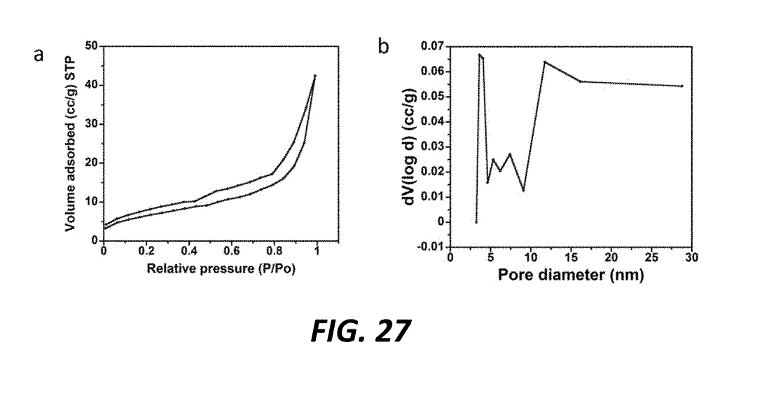

FIG. 28 shows a Ragone plot of a brGO/Mb supercapacitor in an acid, showing high energy density up to 1.1 Wh/kg at high power density.

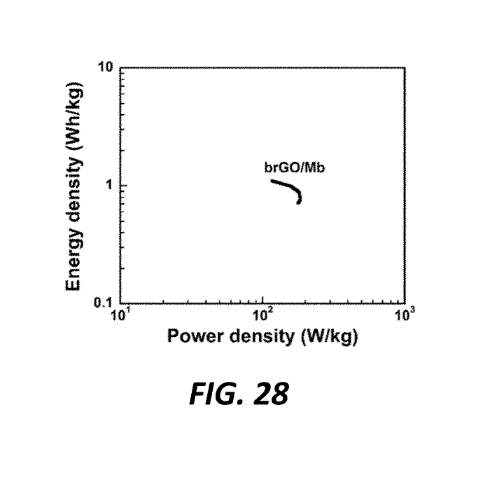

FIG. 29 shows a Trypan blue exclusion test of COS-7 cells subjected to brGO/Mb supercapacitor cycling tests with different numbers of charge-discharge cycles. Images show mainly live cells with a few dead cells stained in blue. (a) Control COS-7 cells were not subjected to the device, showing 8±4 dead cells. A few dead cells normally appeared during the cell culture preparation, and before testing the device. (b) COS-7 cells subjected to 5000 cycles with no significant difference in the number of dead cells compared to the control. (c) COS-7 cells subjected to 10,000 cycles with nearly equal number of dead cells as the control. Scale bar indicates 50 μm; black arrows show examples of dead cells in all images.

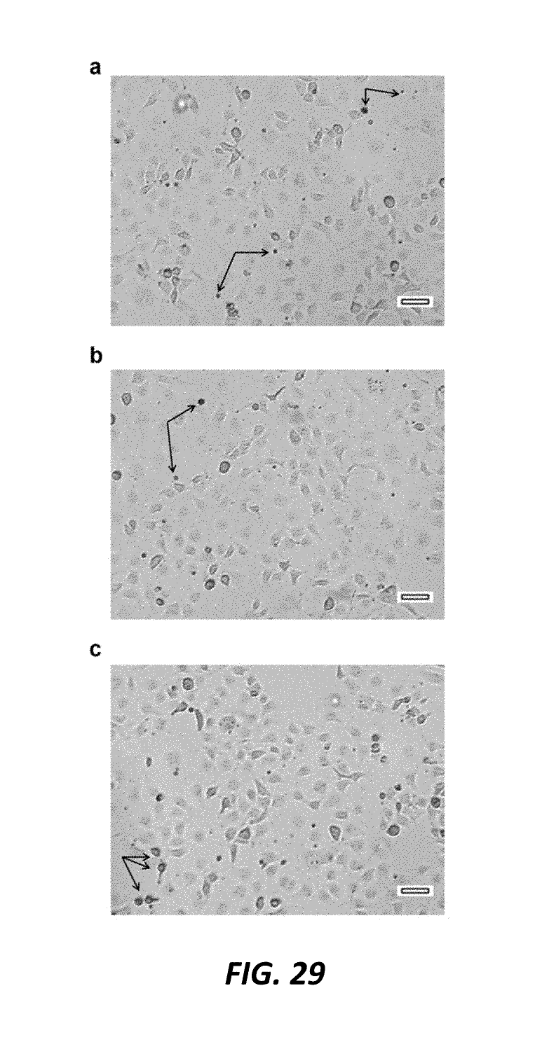

FIG. 30 shows electrochemical impedance spectroscopy (EIS) for a brGO/Mb supercapacitor in biofluids as well as in an acid electrolyte. (a) Nyquist plot with the high frequency region magnified in the inset, showing nearly identical impedance behavior for the device in different electrolytes. The brGO/Mb EC exhibited low series resistance in acid (4Ω) compared with biofluids (8 to 10Ω). (b) Change of phase angle with frequency. The time constant (τ) was calculated from the frequency (f) at an angle of −45° according to the following relation: τ (seconds)=1/f (Hz) at −45°. Inset shows an LED powered by three brGO/Mb ECs connected in series with calf serum saturated separators.

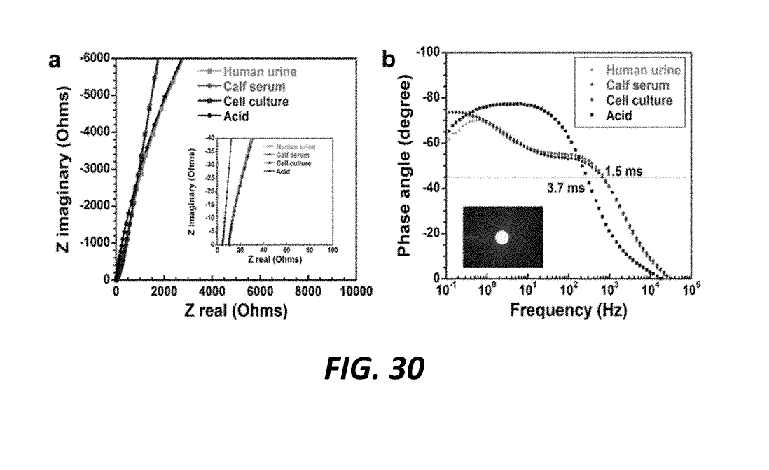

FIG. 31 shows self-discharge behavior of a brGO/Mb EC in calf serum electrolyte. The device was first charged at a constant potential of 1 V using amperometry, followed by an open circuit potential experiment to monitor drop in potential with time.

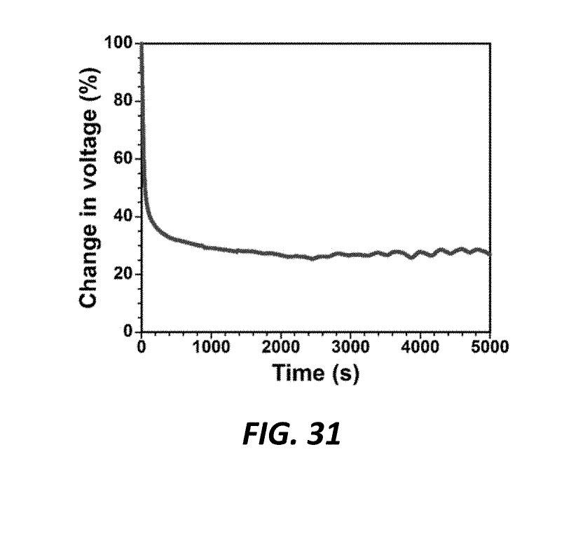

FIG. 32 shows a structural analysis of proteins in relationship to protein capacitive performance in brGO LbL assembled film. (a) Total number of charged amino acid residues (Hist, Lys, Arg, Glu, Asp) in the protein. (b) Total number of charged amino acid residues divided by the molar mass of the corresponding protein. (c) Total number of charged amino acid residues divided by the mass of the film (assuming a mass of 5 μg of protein film).

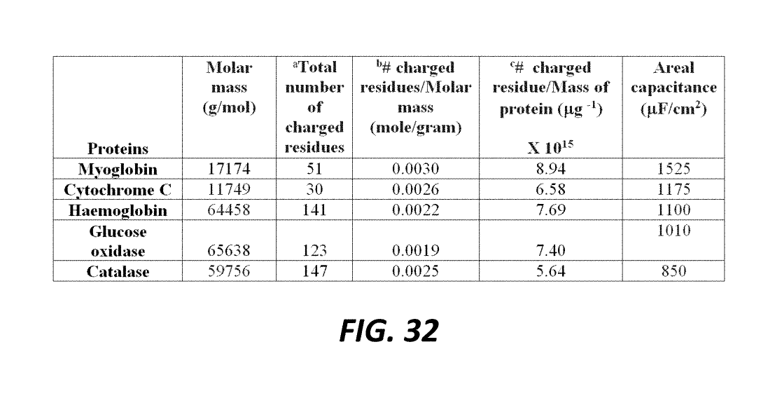

FIG. 33 shows areal and gravimetric capacitance as well as the mass calculated from quartz crystal microbalance for different layers of brGO/Mb film and support materials.

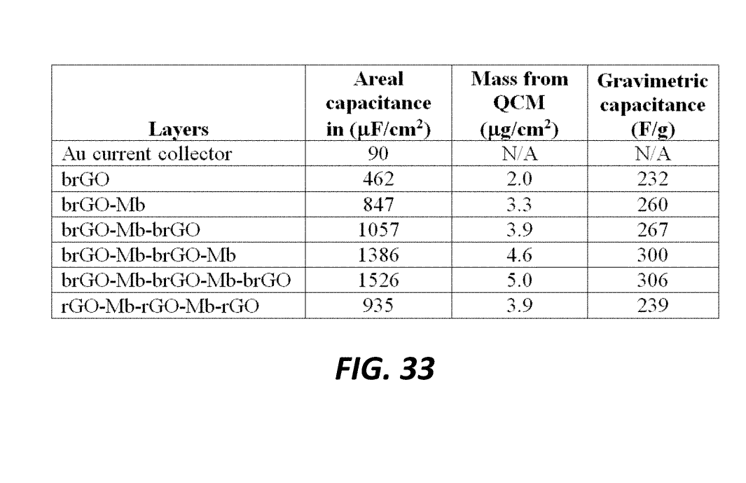

FIG. 34 shows toxicity of bGO/Mb versus as-prepared GO. (a)-(c) Optical microscopy images of COS-7 cells; scale bar is 10 (a) Healthy control COS-7 cells without addition of any graphene-based materials. (b) COS-7 cells co-incubated for 48 hours with 1600 μg/mL protein modified graphene oxide (bGO/Mb) showing no adverse effects on cells compared to control. (c) COS-7 cells co-incubated for only 8 hours with 25 μg/mL of as-prepared GO showing severe cell damage. (d) Dose-dependent toxicity in COS-7 and MEF cells co-incubated with GO for 8 hours, measured in terms of intracellular dehydrogenases (IDH) activity. (e) No significant cell toxicity was observed in COS-7 and MEF cells co-incubated with bGO/Mb as measured by IDH activity even at very high concentrations.

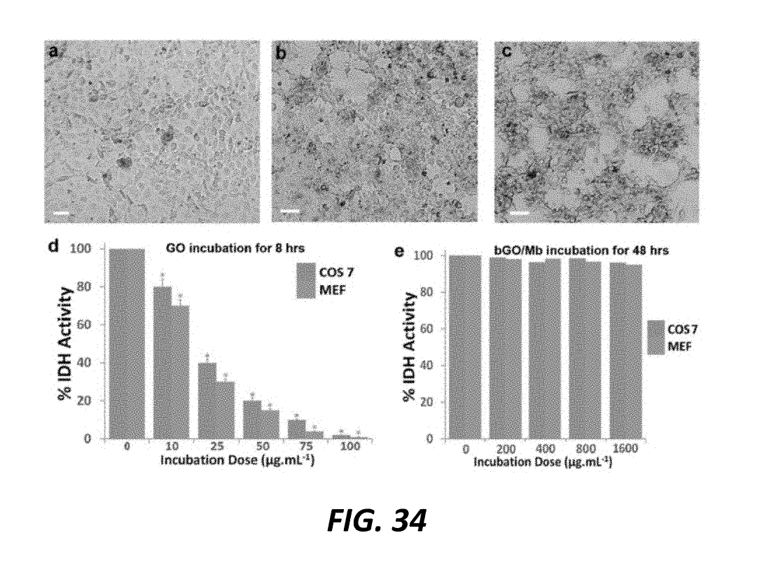

FIG. 35 shows CVs of brGO/Mb bEC showing (a) the effect of bending a device to different angles, (b) the effect of charging/discharging a device to different numbers of cycles while the device is bent to an angle of 90°, and (c) the effect of manually bending a brGO/Mb device to different bending cycles. The device was bent to an angle of 90° in each cycle. (d) The electrochemical performance of brGO/Mb bEC in packageless and PDMS-packaged platforms bent to a 90° angle.

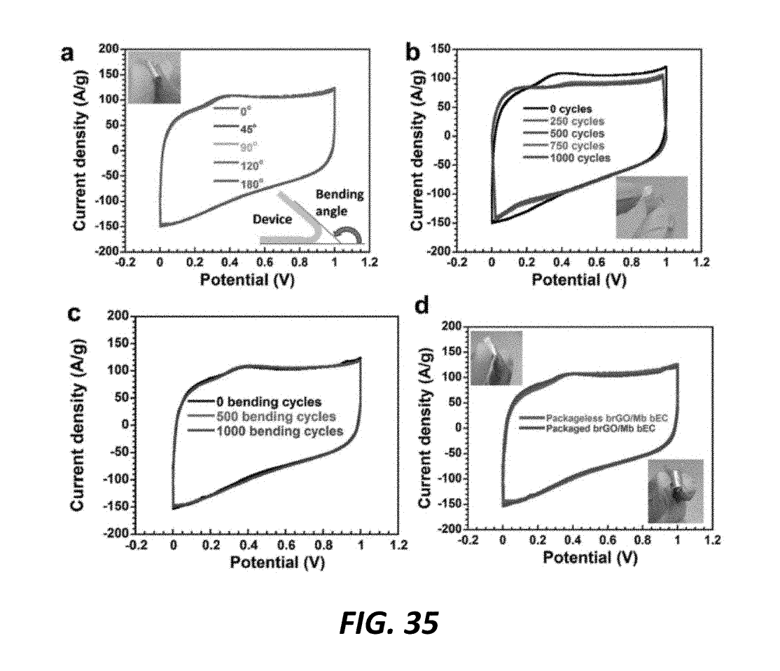

FIG. 36 shows a comparison of the charge/discharge cycles (left) and cyclic voltammogram (right) of both bare capacitors and biosupercapacitors fabricated by direct electrodeposition of graphene oxide-myoglobin composites, demonstrating an increase in capacitive behavior of almost 100 times.

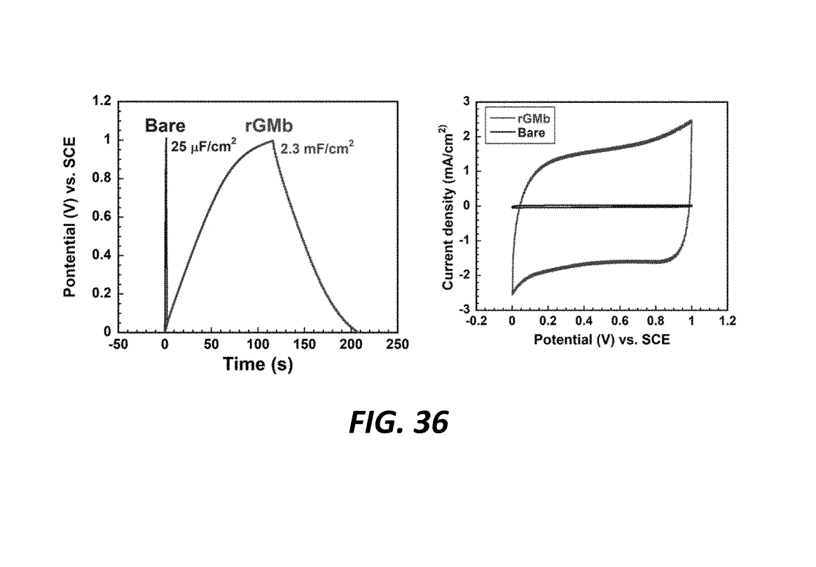

FIG. 37 shows a Wireless Photorechargeable biosupercapacitor. A solar panel converts solar energy to electrical current, which passes through the transmission coil and then gets transferred to a receiver coil. This in turn charges the supercapacitor to power a portable pipette tip biosensor. The tip sensor is a 3D-printed transparent device with a graphite sheet as the working electrode, Pt as a counter electrode, and a reference electrode of Ag/AgCl. The sensor is designed to fit into a 50 mL pipette tip and to receive power from the portable biosupercapacitor attached to it.

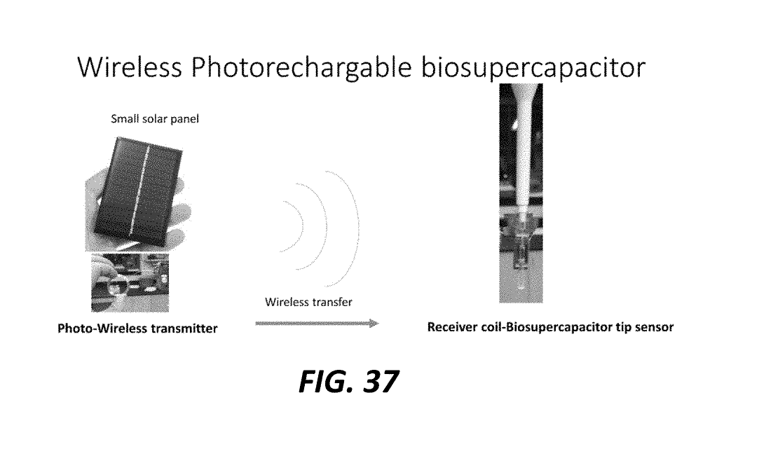

FIG. 38 shows electrochemiluminescence signals from a pipette tip sensor after applying a potential of 1.25 V using a photorechargeable biosupercapacitor. Results show the effect of different aflatoxin-1 (AFB-1) concentrations on DNA damage, which was detected in terms of ECL signal increase driven by the photorechargeable biosupercapacitors.

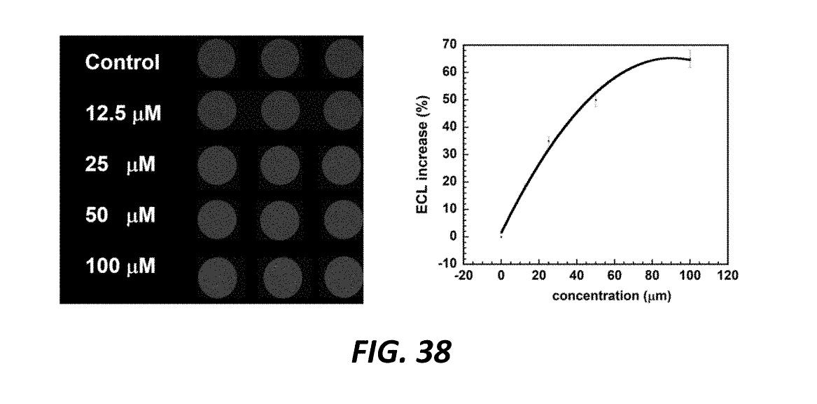

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

In one aspect, the invention relates to supercapacitors, particularly implantable supercapacitors fabricated by rational design and synthesis of new heterostructures in which two-dimensional graphene is combined with proteins, such as bovine proteins. These graphene-protein based devices are also referred to herein as bioelectrochemical capacitors (bECs), biological supercapacitors, and biosupercapacitors.

Among other applications, these devices could replace batteries in next generation implantable biomedical devices, such as cardiac and gastric pacemakers; deep brain, bladder, and bone stimulators; automated drug delivery systems; artificial vision; and biosensors. In the United States alone, at least 2.9 million patients use pacemakers to help resolve medical problems, and the number of patients in need of a pacemaker is expected to increase dramatically each year.

Like other electronic devices that rely on batteries for their power, currently implantable medical devices must be replaced when the battery is drained. This necessitates that the patient undergo painful surgery at significant expense multiple times in a lifetime. Having supercapacitors as power sources for pacemakers instead of batteries will increase the life expectancy of pacemakers to extend to a patient's lifetime. This avoids patients' painful surgeries as well as high costs. Also, supercapacitor-powered pacemakers are safer than those powered with batteries.

Additionally, the self-powered supercapacitor/harvester system disclosed herein may provide a power source for portable electronics such as cell phones, laptops, tablets, and other battery powered electronics.

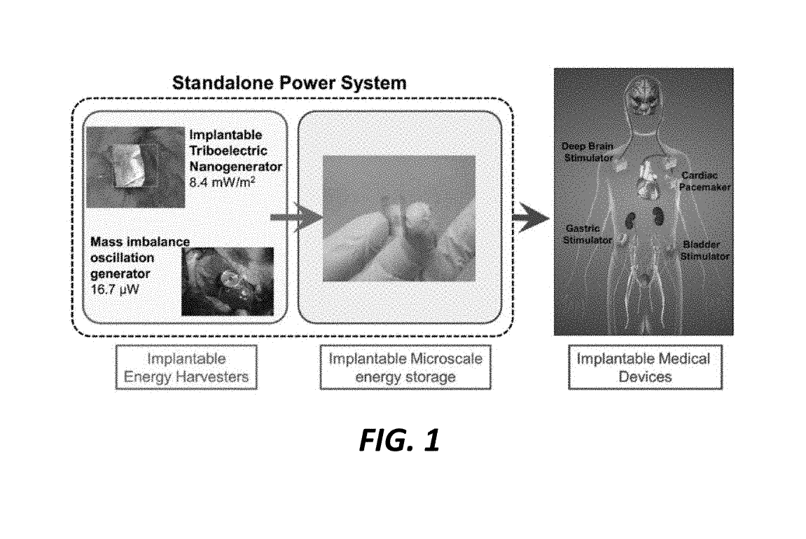

In certain embodiments, the disclosed supercapacitors are only one micrometer thick, achieving high energy density comparable to that of lithium thin film batteries, but with much higher power density and faster discharge of signal. These devices may safely utilize actual body fluids as electrolytes for operation with a very long life cycle. The disclosed supercapacitors showed no detectable signs of cell toxicity when exposed to mouse embryo fibroblasts and COS-7 cell cultures for 5,000 charge-discharge cycles. Coupling the microscale graphene-protein supercapacitor with a standalone power source could power the next generation of miniaturized implantable devices for the lifetime of a patient. Such a standalone power source may be an implantable energy harvester, as illustrated in FIG. 1. A suitable implantable energy harvester may be piezoelectric or thermoelectric, for example. In certain embodiments, the implantable energy harvester is an implantable triboelectric nanogenerator. In certain other embodiments, the implantable energy harvester is a mass imbalance oscillation generator.

In certain embodiments, the disclosed supercapacitors provide very high energy density comparable to Li thin film batteries, but with much higher power density. For example, ultrathin brGO/Mb supercapacitors described herein achieved ultrafast frequency response (cell time constant 1.5 milliseconds), making these devices perfect for the fast discharge of electrical signal to the desired organ by next generation pacemakers as well as acting as AC rectifiers, and the ability to use body fluids as electrolyte for operation without any toxic effects to neighboring cells.

Supercapacitors are high-performance electrochemical capacitors (ECs) that store energy at much higher power density than batteries. Power systems for implantable devices require an intermediate energy storage system, such as ECs, for their operation. By storing generated energy in an EC, output power can periodically drive existing medical implants. However, there are some stringent requirements for implantable medical devices. They must be safe during operation and have predictable performance and high reliability. They should provide service over many years with no maintenance required. It is also important that they have high volumetric energy density to enable the miniaturization of the entire implanted system (FIG. 1).

Compared to batteries, ECs have faster charge-discharge rates, lower internal resistance, higher power density, better cycling stability, and the ability to use external fluids as electrolytes. These devices have the potential to power a new generation of implantable devices, such as cardiac and gastric pacemakers; deep brain, bladder, and bone stimulators; automated drug delivery systems; artificial vision; and biosensors. However, ECs are currently limited by relatively low volumetric and gravimetric capacitances, and low energy densities (energy stored per unit volume or mass) that are less than those of batteries. In addition, batteries in general are considered the least “green” component in any electronic device. They often use toxic materials and operate on liquid electrolytes, which can be very harmful if leakage occurs. Using such batteries for implantable medical devices not only make them bulky but also raises safety concerns. Thus, rationally designing green and sustainable bioelectrochemical capacitors (bECs), also called biosupercapacitors, with high volumetric energy density is highly desirable. It is also important that these bECs use electrode materials and electrolytes that are safe to living cells.

Graphene sheets feature single layers of carbon atoms with unique electrical properties that are promising for designing ultrathin bECs and other novel technological applications. It is an attractive nanomaterial for implantable bECs, given its high theoretical capacitance of 550 F/g and its rich surface chemistry, which enables further processing into composite materials with desirable properties.

The inventors have surprisingly developed graphene-based bECs with one or more, preferably all, of the attractive qualities described above by, for example, attaching polyamine-derivatized (cationized) bovine serum albumin (cBSA) to the surface of graphene oxide (GO) as a nanospacer and further deposition of heme protein myoglobin in a layer-by-layer (LbL) process, followed by electrochemical reduction. Other cationized and small-heme proteins may be used, however, and other processes besides LbL, as described herein.

In certain embodiments, disclosed are graphene-based bECs having an ultrahigh volumetric capacitance in biofluids up to 655 F/cm³ at a scan rate of 100 mV/s, and 534 F/cm³ at a current density of 2.5 A/g. Energy density may reach up to 1.8 mWh/cm³, which is 3 to 11 times higher than commercially available thin-film ECs. They may be thinner (1.14 μm in certain embodiments) than a human hair and can provide high power density.

Unlike pristine graphene, which may pose potential risks to human cells, the new hybrid material disclosed herein shows no signs of cell toxicity at concentrations 160 times higher than those used in a typical embodiment of the disclosed bECs. In certain embodiments, the encapsulated device showed no adverse effects on cells after 4 days of continuous charge/discharge cycles.

Disclosed herein, among other things, is a new platform for the design of ultrathin bECs utilizing for the first time human biofluids as electrolytes for the development of, for example, the next-generation implantable medical devices (FIG. 1).

The disclosed invention includes a novel group of implantable supercapacitors—energy storage devices. New heterostructures are fabricated by rational design. In certain embodiments, two-dimensional graphene is combined with proteins. In certain embodiments, the proteins are bovine proteins.

These devices may replace batteries in next generation implantable biomedical devices, such as cardiac and gastric pacemakers; deep brain, bladder, and bone stimulators; automated drug delivery systems; artificial vision; and biosensors, for example. Unlike batteries, the disclosed devices have the potential to power all implantable medical devices for the lifetime of a patient. Additionally, the specifications for energy density and power density far exceed industry standards, while also being completely compatible with biological systems.

In contrast to conventional batteries, the disclosed supercapacitors may be only one micrometer thick and achieve high energy density comparable to that of lithium thin film batteries, but with much higher power density and faster discharge than lithium thin film batteries. Additionally, the disclosed devices may be able to safely utilize actual body fluids as electrolytes for operation, with very long life cycle. The disclosed supercapacitors show no detectable signs of cell toxicity when exposed to mouse embryo fibroblasts and COS-7 cell cultures for 5000 charge-discharge cycles.

Coupling of microscale graphene-protein supercapacitors with implantable energy harvesters may power the next generation of miniaturized implantable devices for the entire lifetime of a patient. Currently, implantable medical devices must be replaced when the battery is drained, which necessitates that the patient undergo painful surgery at significant expense multiple times in a lifetime. Instead, having the disclosed supercapacitors as power sources for pacemakers, for example, may increase the life expectancy of pacemakers to extend for a patient's lifetime. This avoids patients' painful surgeries as well as very high costs. Also, supercapacitor powered pacemakers may be safer than those powered with batteries.

The disclosed supercapacitors may provide high energy density with high power density. The high energy density may be comparable to Li thin film batteries. The high power density may exceed Li thin film batteries. The disclosed supercapacitors may achieve ultrafast frequency response. For example, the frequency response may be characterized by a cell time constant of 1.5 milliseconds. A fast discharge of electrical signal may be desirable in pacemakers or AC rectifiers, for example. The disclosed supercapacitors may be able to use natural body fluids as electrolyte for operation. They may also be able to operate without any toxic effects to neighboring cells. In some embodiments, the supercapacitor may be 1 micrometer thick.

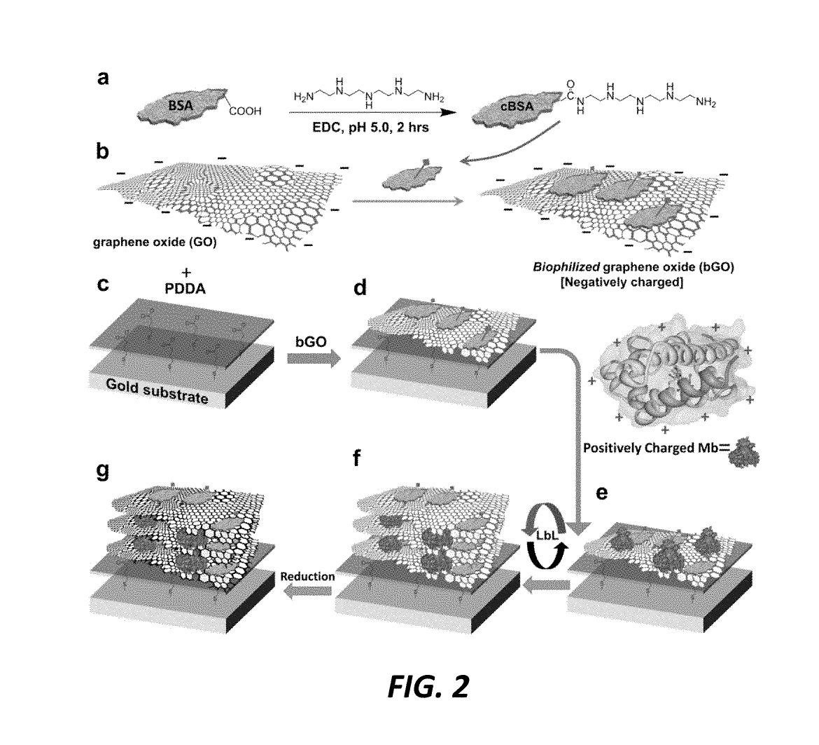

FIG. 1 illustrates a scheme of a possible application of the disclosed supercapacitors (central panel) as micro-power sources for biomedical implants. FIG. 2 illustrates one method of fabricating the disclosed electrodes.

The disclosed supercapacitors were designed by a bottom up approach for the assembly of graphene-based electrodes (FIG. 2). The supercapacitors may be fabricated using bGO, a novel biophilized graphene oxide (bGO) nanocomposite featuring graphene oxide sheets (GO) modified with cationized bovine serum albumin (cBSA). See Pattammattel, A. et al., Tuning the Activities and Structures of Enzymes Bound to Graphene Oxide with a Protein Glue. Langmuir 29, 15643-15654 (2013), incorporated herein in its entirety.

Layer-by-Layer (LbL) deposition (see Lvov, Y. Electrostatic Layer-by-Layer Assembly of Proteins and Polyions, In Lvov, Y., Möhwald, L. Protein Architecture: Interfacing Molecular Assemblies and Immobilization Biotechnology, Marcel Dekker, 125-167 (2000), incorporated herein in its entirety) may be used to fabricate electrodes of alternate layers of bGO and myoglobin (Mb) onto a 1.0 cm² ultrathin gold sheet to form several bGO/Mb bilayers.

Then bGO/Mb may be electrochemically reduced to brGO/Mb, where brGO is reduced biophilized graphene oxide (FIG. 2 at (g)). A film of three layers of brGO alternated with two layers of Mb gave the largest capacitance per unit volume, up to 860 F/cm³ at 25 mV/s. In this measurement, the contribution of a current collector and separator was subtracted, as described below. EC devices made from these electrodes were able to utilize cell culture media and mammalian biofluids as electrolytes for high capacitance output with no measurable signs of cytotoxicity. The cytotoxicity measurements were in cell culture media after 5,000 charge/discharge cycles.

The disclosed brGO/Mb electrodes may be fabricated by reacting a surface of a thin gold current collector with 5 mM mercaptopropionic acid in 70% ethanol to self-assemble an anionic alkylthiol monolayer. Next, poly(diallyldimethyl ammonium) chloride (PDDA) may be adsorbed from an 4.0 mg/mL solution in 0.5 M NaCl onto the gold surface to form a cationic hydrophilic/hydrophobic underlayer suitable for adsorbing the next layers. A negatively charged layer then may be adsorbed from 0.7 mg/mL bGO in pH 7.2 buffer, followed by a layer of Mb adsorbed from 3.0 mg/mL solution, pH 5.0. In this way, three layers of bGO may be sandwiched between two layers of myoglobin, for example, and named as bGO/Mb film. Electrochemical reduction of the bGO/Mb film may be performed using amperometry at a constant potential of −1.2 V for 70 s in 10×10⁻³ M acetate buffer, pH 5.5 in 0.5 M salt, such as KBr or lithium perchlorate (LiCO4), and the resulting film may be named as brGO/Mb.

Without being limited to theory, graphene may be made more compatible with living cells by cBSA and Mb. They may also provide a good source of heteroatoms to improve the specific capacitance of graphene by introducing pseudocapacitive behavior. See Wu, Z.-S. et al. Layer-by-Layer Assembled Heteroatom-Doped Graphene Films with Ultrahigh Volumetric Capacitance and Rate Capability for Micro-Supercapacitors. Adv Mater 26, 4552-4558 (2014), incorporated herein by reference in its entirety. The proteins may also act as polyionic nano-spacers to prevent agglomeration and restacking of graphene, which can undermine electrochemical performance. A gold current collector may be employed, because gold is among the safest materials for medical application. See Shukla, R. et al. Biocompatibility of Gold Nanoparticles and Their Endocytotic Fate Inside the Cellular Compartment: A Microscopic Overview. Langmuir 21, 10644-10654 (2005), incorporated herein by reference in its entirety.

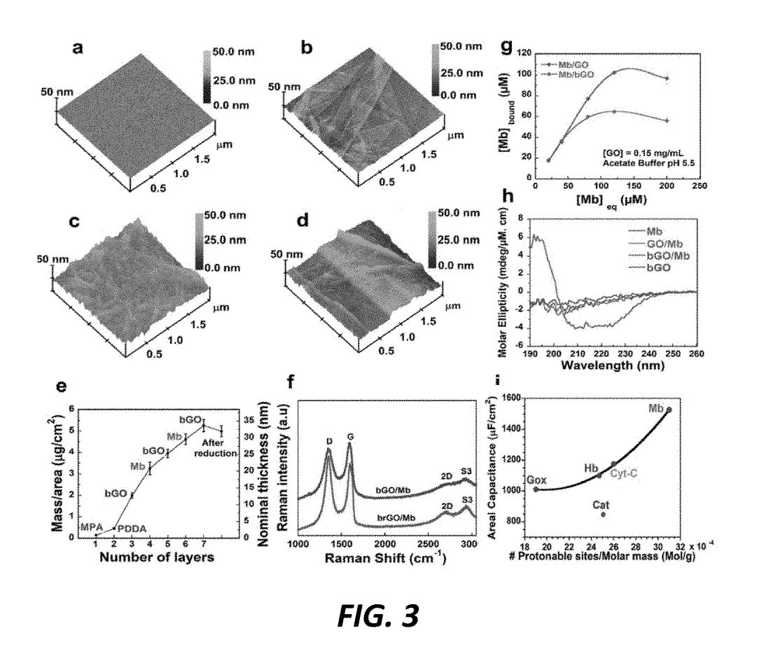

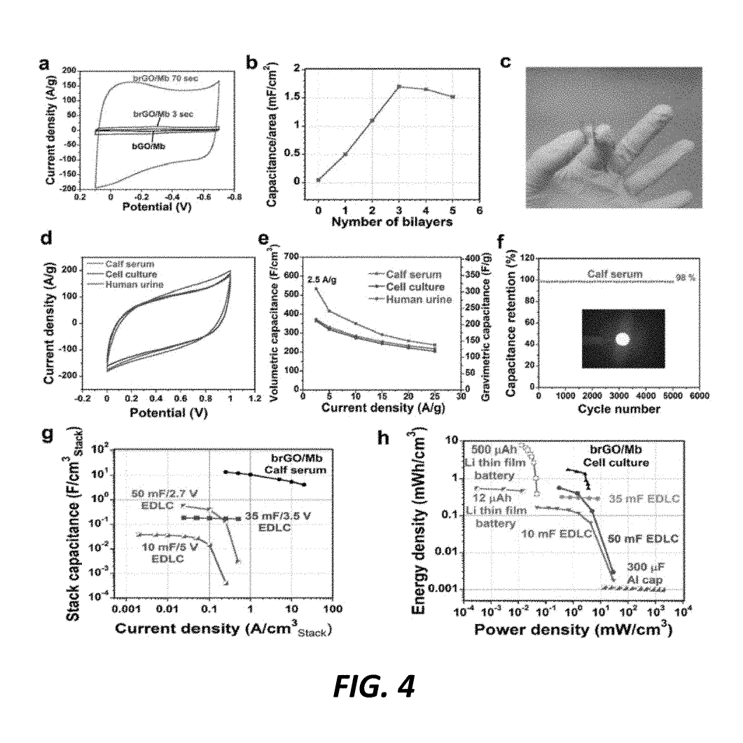

FIG. 3 illustrates various characterization results for certain embodiments of the disclosed graphene-protein electrodes. FIG. 4 illustrates electrochemical behavior of a brGO/Mb supercapacitor according to an embodiment of the disclosed invention.

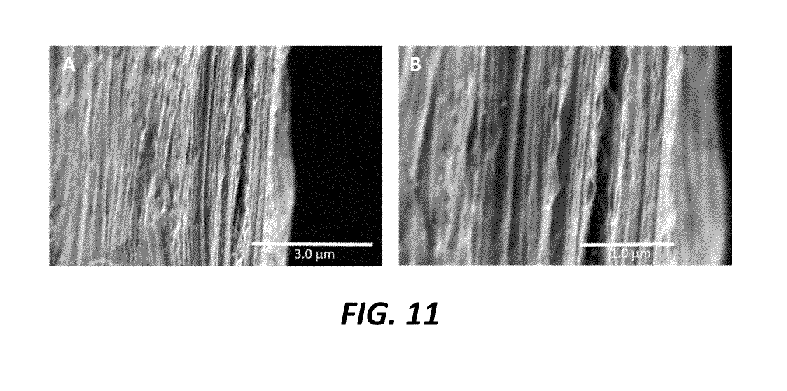

In certain embodiments, the film was electrochemically reduced in 10 mM acetate buffer, pH 5.5 containing 0.5 M KBr at constant potential of −1.2 V vs. SCE for 70 s. The reduction of bGO in the film to brGO was indicated by the change of film color from brown to black, and was also supported by a reduction peak for bGO found at −0.95 V vs. SCE by cyclic voltammetry (CV) that disappears upon subsequent scans (FIG. 11 at (a)). In addition, a dramatic increase in capacitance was found after bGO reduction to brGO (FIG. 4 at (a), FIG. 11 at (b-c)). CV profiles showed characteristic Fe^{III}/Fe^{II} peaks of Mb at −0.3 V vs. SCE in acetate buffer; the characteristic peaks appeared before and after reducing the film for 3 s, but continued reduction for 70 s caused a large increase in the charging current and loss of the Mb peaks (FIG. 4 at (a)).

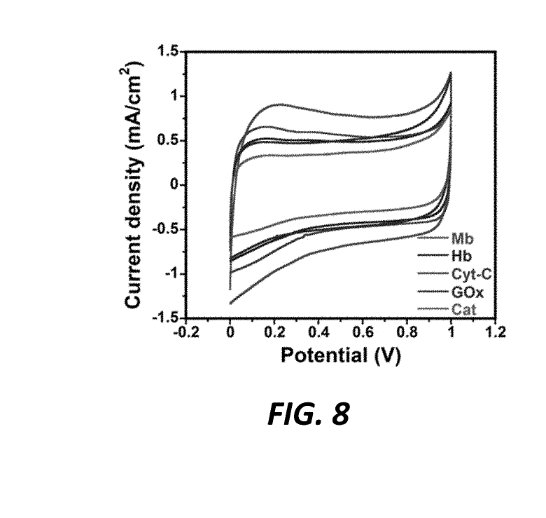

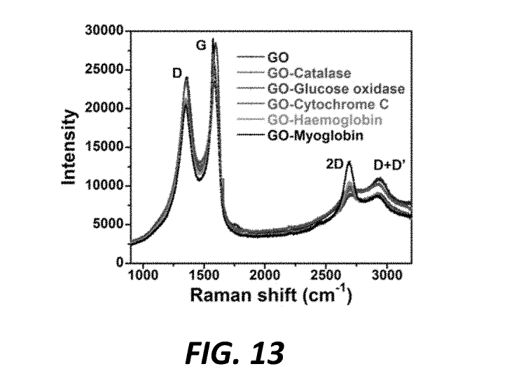

A photograph of one embodiment of an implantable supercapacitor according to the disclosed invention is shown in FIG. 4 at (c). It was assembled from two brGO/Mb/brGO/Mb/brGO electrodes bound to a flexible thin gold foil with a thickness of 42±8 nm (FIG. 8), obtained after etching away a plastic protective layer of a gold compact disc (CD). To get a neat and ultrathin supercapacitor, a layer of polyvinyl acetate gel saturated with electrolyte was loaded onto the electrodes to serve as both the separator and electrolyte with an average separator thickness of 992 nm (FIG. 13), which may have simplified the processing of the electrodes into paper-like flexible supercapacitors. The total thickness of the device was only 1.14 μm, providing a thin, lightweight power source for implantable devices, for example. Despite being ultrathin, the supercapacitor demonstrated excellent mechanical properties; the device could be bent, folded, and twisted several times without cracking, for example.

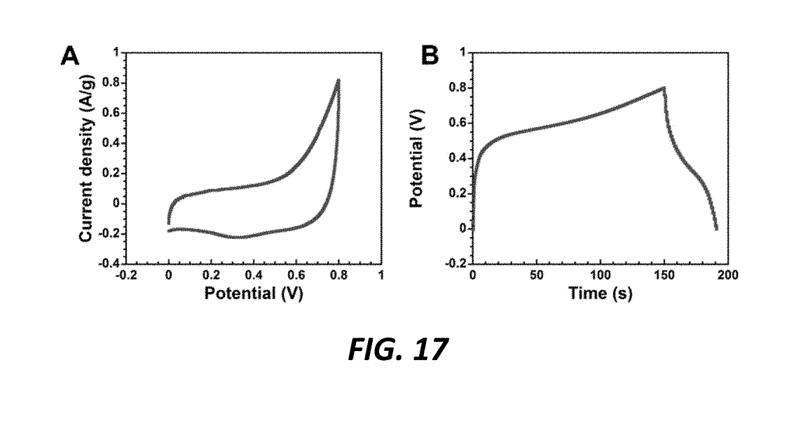

The overall performance of a brGO/Mb supercapacitor is displayed in a Ragone plot (FIG. 17). The brGO/Mb supercapacitor displayed a high energy density of 1.1 mWh/g at a high power density of 13.5 W/g, calculated based on the total mass the device stack as illustrated in FIG. 17. A similar plot using the volume of the device stack is shown in FIG. 4 at (h). For comparison, the energy density and power density of a 300 μF/3 V aluminum electrolytic capacitor, 12 μAh/3.3 V Li thin film battery, 500 μAh/5 V Li thin film battery, and three commercially available EDLCs designed for small scale applications were also tested. The brGO/Mb supercapacitor can provide a high energy density of 1.8 mWh/cm³, which is comparable to Li thin film batteries, and 3 to 11 times higher than thin film commercial EDLCs. The brGO/Mb device can also deliver a power density of 5 mW/cm³, which may be sufficient to drive all implantable medical devices currently known (FIG. 4 at (h)).

A performance comparison with conventional thick film electrodes is irrelevant, as thick film electrodes are not suitable for miniaturized implantable devices.

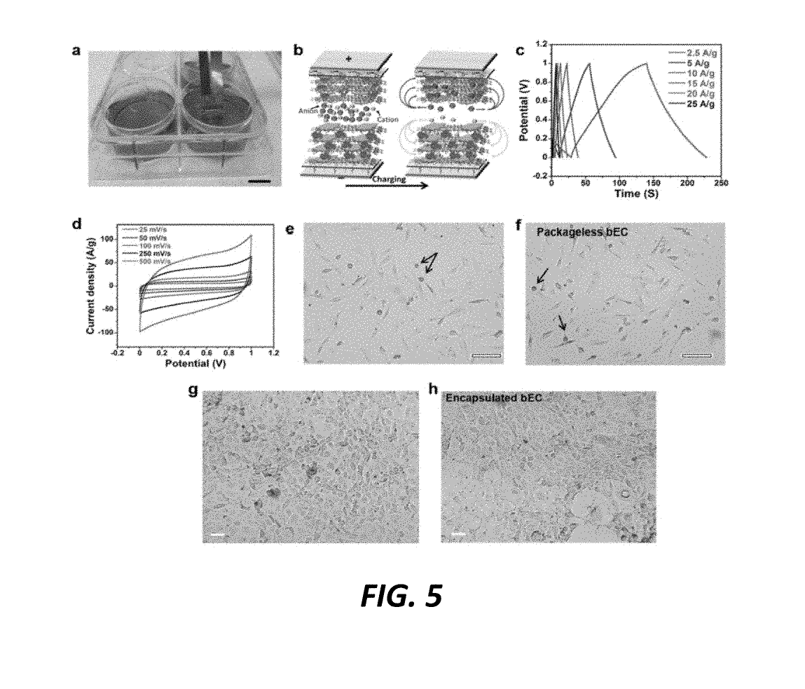

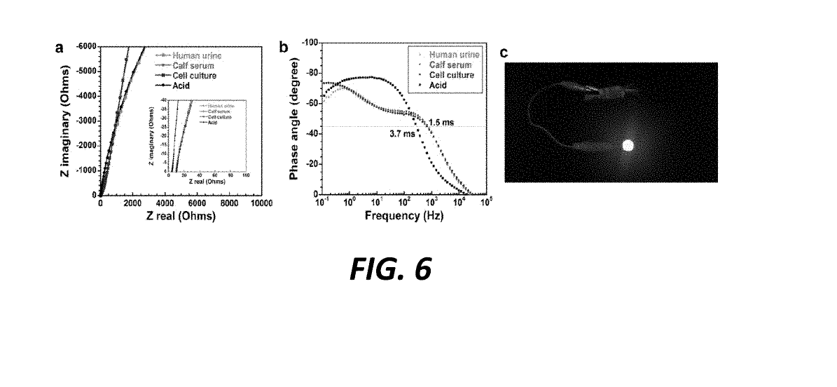

FIG. 5 illustrates the performance of a brGO/Mb supercapacitor in cell culture medium. FIG. 6 illustrates results of electrochemical impedance spectroscopy (EIS) for a brGO/Mb supercapacitor in biofluids and in acid electrolyte.

Electrochemical impedance spectroscopy (EIS) showed nearly straight lines in the high frequency region in certain tested electrolytes (FIG. 6), for example, all tested electrolytes. A brGO/Mb device showed a very fast frequency response as measured by the frequency at −45° (FIG. 6 at (b)). For example, the very fast frequency response may be characterized by a cell time constant of 1.5 ms in biofluids and 3.7 ms in acid. Additionally, the observed phase angle close to −90° suggests nearly ideal capacitive behavior. Three supercapacitors made up of the ultrathin brGO/Mb films were capable of powering a light emitting diode utilizing calf serum as the electrolyte (FIG. 6 at (c)).

Without being limited to theory, the fast frequency response of the devices is likely to be related to fast ion movement through the thin electrode assemblies, with myoglobin possibly acting as a nanochannel for fast ion transport. See Levantino, M. et al. Ultrafast myoglobin structural dynamics observed with an X-ray free-electron laser. Nat Commun 6 (2015) and Jiang, Y., Kirmizialtin, S. & Sanchez, I. C. Dynamic void distribution in myoglobin and five mutants. Sci rep 4, 4011 (2014), incorporated herein by reference in their entireties.

### Construction of Ultra-Thin Gold Current Collectors from Gold CD

A gold compact disc (CD) was treated with 70% nitric acid in a Petri dish for 1 to 2 minutes to remove the outer polymeric layer, thus exposing a thin gold layer with a median thickness of 42±8 nm, as measured by focused ion beam scanning electron microscope (FIB-SEM) (FIG. 8). The gold layer was washed with 70% ethanol and then water to remove any nitric acid contamination; this produced a clean thin gold current collector in a rigid plastic support. For a flexible current collector, the gold layer was removed from the hard plastic support under a stream of running water focused at the edge of the CD, then dried and transferred to the sticky side of very thin transparent tape, washed with DI water, and then dried before use. The gold layer was then cut into pieces (1.0 cm² each, measured using a Varnier Calliper) and then incubated into 5 mM mercaptopropionic acid in 70% ethanol to produce an anionic monolayer of HOOC-alkylthiol with the carboxylic groups directed towards the surface, ready for binding the next cationic layer.

### Assembly of brGO/Mb Electrode Material

First, biophilized graphene oxide (bGO) was synthesized by the method described in A. Pattammattel, M. Puglia, S. Chakraborty, I. K. Deshapriya, P. K. Dutta, C. V. Kumar, Langmuir 2013, 29, 15643-15654, hereby incorporated by reference in its entirety. Briefly, bovine serum albumin (BSA) and 2-ethandiamine were mixed together, followed by a slow addition of crosslinking agent to avoid excessive crosslinking between BSA molecules. See M. J. Novak, A. Pattammattel, B. Koshmerl, M. Puglia, C. Williams, C. V. Kumar, ACS Catalysis 2016, 6, 339-347, hereby incorporated by reference in its entirety. Chemically modified bovine serum albumin (cBSA) was allowed to bind to GO through controlled addition of cBSA to GO with continuous stirring for 20 hours to prepare bGO.

A layer-by-layer assembly technique was used to alternate oppositely charged layers on the gold current collector to construct the electrode material. Poly(diallyldimethyl ammonium) chloride (PDDA) (4 mg/mL in 0.5 M NaCl) was adsorbed for 20 min to the gold surface to assemble a cationic polyion polymeric layer. Adsorption of each layer was followed by washing with DI water and drying with nitrogen. A negatively charged bGO (0.7 mg/ml in 10 mM phosphate buffer, pH 7.2) was then adsorbed for 30 min, followed by a positively charged myoglobin (3 mg/mL in 10 mM acetate buffer, pH 5.5) adsorbed for 30 min, and in this way, three layers of bGO were sandwiched between two layers of myoglobin, named as a bGO/Mb film.

Electrochemical reduction of the bGO/Mb film was then performed using amperometry at a constant potential of −1.2 V for 70 seconds in 10 mM acetate buffer, pH 5.5 in 0.5 M KBr, and the resulting film was named brGO/Mb. A pyrolytic graphite electrode in a three-electrode system was used for the selection of the best protein (Mb), the number of bilayers (three), and for characterizing the electrochemical reduction step.

In certain embodiments, brGO-protein supercapacitors were fabricated with high volumetric capacitances of up to 860 F/cm³. Proteins in these devices not only made them compatible with cell-containing environments, but also provided pseudocapacitance as heteroatom rich nanostructures and may act as nanochannels for shuttling ions of the biofluids to conductive graphene interlayers. These devices retained high energy density comparable to that of Li thin film batteries, while utilizing common biofluids or cell media as electrolytes to power LED with little loss of performance and low cell toxicity. Unlike batteries, which suffer from relatively slow discharge rates, the ultrathin brGO/Mb supercapacitors achieved ultrafast frequency response (cell time constant 1.5 ms), making these devices advantageous for fast discharge of electrical signal to a desired organ by next generation pacemakers, for example, as well as for acting as AC rectifiers. With protein as a major component in certain embodiments, these devices do not release toxic components.

The disclosed supercapacitors may be integrated into internally-powered biodevices. The disclosed supercapacitors may also be integrated into other devices.

### A Highly Scalable Method for Fabrication of Graphene Protein Biosupercapacitors

Positively charged protein was added to a solution of negatively charged graphene oxide slowly with stirring. This was followed by introduction of a bare current collector to the solution and application of −1.1 V vs. SCE, which lead to formation of uniform films of thick protein with reduced graphene oxide, such as reduced graphene/myoglobin (rGMb), for example. This method lead to thicker films than traditional layer by layer assembly methods. This preferred method also lead to construction of high performance biosupercapacitors in 30 seconds of fabrication time, which is scalable and suitable for commercial production.

### Device Fabrication

A thin water-insoluble polyvinyl acetate (PVA) separator with an average thickness of 992 nm (FIG. 13) was sandwiched between two brGO/Mb electrodes in a bottom up designed supercapacitor and wrapped with Kapton tape. PVA was first diluted in test electrolyte solution to a concentration of 1 to 5%. Adjustments may be needed until achieving a full intact thin layer. The diluted suspension of PVA was then applied to one side of the supercapacitor, left to be just partially wet, and then the device was assembled. In this exemplary embodiment, the full stack thickness of the device was 1.14 μm including electrodes, current collectors, and separators. The device was then dipped into the testing electrolyte for 10 minutes before electrochemical performance was evaluated, to ensure wetting of the separator and the electrodes with the test electrolyte. The device was connected to an electrochemical workstation through extension from a gold collector via alligator clips, while copper tape was used to connect the device to the electrochemical workstation when using cell culture media as electrolyte, to avoid possible contamination of the workstation alligator clips by the media.

As known in the art, polyvinyl alcohol and polyvinyl acetate are both abbreviated “PVA”. However, although polyvinyl alcohol is a very common material for separators in supercapacitor fabrication, it was typically avoided because of its toxicity as well as solubility in aqueous electrolytes, which may result in dissolving the separator during testing, unlike polyvinyl acetate.

In certain embodiments, a midstream urine sample was collected from a healthy male individual and used as an electrolyte.

### Contribution of Current Collector to the Device Capacitance

The contribution of the gold CD as a current collector to the device capacitance was evaluated by comparing the capacitance/area of the device with bare gold CD pieces (without brGO/Mb film) with a device with a brGO/Mb film in acid electrolyte at low and high scan rates. At a low scan rate of 50 mV/s, the capacitance of the device with bare gold was 50 pF/cm², compared to 1100 μF/cm² for the brGO/Mb supercapacitor, while at a high scan rate of 1000 mV/s, the capacitance of the device with bare gold was 32 μF/cm², compared to 608 μF/cm² for the brGO/Mb supercapacitor. This very low contribution of the gold current collector is equivalent to only 4.5% and 5% at the low and high scan rate, respectively. The contribution of the current collector was subtracted from the capacitance of the electrode inter alia. Moreover, the capacitance of the bare Au electrode with PDDA adsorbed on the surface was 90 μF/cm² in calf serum at a current density of 0.005 mA/cm², which represents 6% of the areal capacitance of the final brGO/Mb electrode. The contribution of the Au-current collector to the areal capacitance was subtracted from the capacitance in the charge/discharge curves of the different electrode materials disclosed herein.

### Capacitance Calculations

The specific capacitance of a device was calculated from galvanostatic charge/discharge curves using the following relation:

$${C}_{device} = \frac{{i}_{app}}{- Δ E / Δ t} \qquad ( 1 )$$

where iₐₚₚ is applied current in Amperes and −ΔE/Δt is the slope of the discharge curve.

The gravimetric capacitance of the device (C_{s(device)}) and the stack volumetric capacitance (C_{v(stack)}) were calculated using the following equations:

$$\begin{matrix}{C}_{s ( device )} = \frac{{C}_{device}}{M} & ( 2 ) \\ {C}_{v ( stack )} = \frac{{C}_{device}}{V} & ( 3 )\end{matrix}$$

where M is the total mass of the active films on both electrodes and V is the total volume of the device, including flexible supports, current collectors, active films, and separator (but not packaging). Capacitance/area was calculated by replacing the volume of the device in Equation 3 with the device area.

The gravimetric capacitance of the electrode (C_{s(electrode)}), and the volumetric capacitance of electrode (C_{v(electrode)}) are given by the following formulas:

$$\begin{matrix}{C}_{s ( electrode )} = {C}_{s ( device )} \times 4 & ( 4 ) \\ {C}_{v ( electrode )} = \frac{2 \times {C}_{device}}{{V}_{electrode}} & ( 5 )\end{matrix}$$

Internal resistance (R) of the supercapacitor was calculated through dividing the voltage drop (V_{drop}) (measured at the beginning of the discharge curve) by the applied current as follows:

$$R = \frac{{V}_{drop}}{2 \times {i}_{app}} \qquad ( 6 )$$

Power density (P) in W/kg and energy density (E) in Wh/kg were measured for the whole device using the following equations:

$$\begin{matrix}P = \frac{E}{t} & ( 7 ) \\ E = \frac{1}{2 M} {C}_{( device )} \times {V}^{2} & ( 8 )\end{matrix}$$

where t is the discharge time of the stacked device, V is the operating potential window for the discharge curve subtracted from V_{drop}, and M is the total mass of the active material of the two electrodes in grams, while C_{s(device)} is the gravimetric capacitance of the device obtained from Equation 2.

The device capacitance was calculated from cyclic voltammograms using the following integral relation:

$${C}_{device} = \frac{{\int }_{{V}_{1}}^{{V}_{n}} i ( {V}_{2} - {V}_{1} )}{υ \times Δ E} \qquad ( 9 )$$

where i, V₁, V₂, υ, and ΔE are the measured current, the starting voltage, the ending voltage, the scan rate, and the operating potential window, respectively.

### Quartz Crystal Microbalance (QCM) Measurements

Gold resonators (0.16 cm²) were used in the experiments. Mass/area was calculated from the frequency shift (ΔF) according to the following equation (see Y. Lvov, K. Ariga, I. Ichinose, T. Kunitake, Journal of the American Chemical Society 1995, 117, 6117-6123, herein incorporated by reference in its entirety):

M/A(g/cm²)=−ΔF_{(Hz)}×1.832×10⁸ (10)

Nominal film thickness (d) was calculated from ΔF due to adsorption of the film on one side of the gold resonator, as shown by the following equation:

d(nm)=−2(0.016)ΔF(Hz) (11)

Nominal film thickness of 3 layers of brGO alternated with two layers of Mb was 32 nm, as measured by QCM. The film thickness according to the above equation was confirmed previously by high-resolution electron microscopy. See M. J. Novak, A. Pattammattel, B. Koshmerl, M. Puglia, C. Williams, C. V. Kumar, ACS Catalysis 2016, 6, 339-347, herein incorporated by reference in its entirety. The film thickness was averaged with findings from focused ion beam SEM (FIG. 8).

### Additional Components

Although certain exemplary embodiments of the disclosed invention are described herein, additional suitable components of the invention are also contemplated.

The current collector may comprise, consist essentially of, or consist of a thin foil of gold, silver, aluminum, platinum, alloys thereof, and/or combinations thereof.

The nanomaterial support may comprise, consist essentially of, or consist of carbon-based nanostructures; transition metal carbides, nitrides, or carbonitrides; boron nitrides; and/or combinations thereof. Suitable carbon-based nanostructures may include graphene, graphene oxide, graphene nanoribbons, graphene nanolobes, nano-carbon, carbon nanotubes, carbon nanofibers, carbon nanodiamonds, carbon nanohorns, carbon nanodots, black carbon, activated carbon, and/or carbon nitride. The term “nano-carbon” may mean any carbonaceous material whose size is between about the thickness of a graphene plane up to a few nanometers (e.g. 3 nm) in at least one spatial dimension. Suitable transition metal carbides, nitrides, or carbonitrides may include MXenes, metal chalcogenides (such as WS₂, MoS₂, WSe₂, or GaSe), semi-metals, superconductors (such as NbS₂ or TaSe₂), and/or topological insulators or thermoelectric materials (such as Bi₂Se₃, Bi₂Te, WTa₂, or TcS₂).

The biomaterial may comprise, consist essentially of, or consist of heme-proteins (such as myoglobin, hemoglobin, or cytochrome C), enzymes (such as catalase, glucose oxidase, peroxidases, transferase, or lyase), non-heme proteins (such as bovine serum albumin), and/or combinations thereof. Non-heme proteins may also include amino acid based biomaterials (such as DNA, RNA, polysaccharides, antibodies, small peptides, or poly-lysine). Proteins may be used in positive, negative, or neutral forms.

The separator may comprise, consist essentially of, or consist of a porous membrane (such as polyethylene, polyvinyl acetate, or polyvinyl alcohol) saturated with a biological electrolyte (such as blood, serum, urine, cerebrospinal fluid, saline, or buffer solution), an organic or inorganic electrolyte, and/or combinations thereof.

The packaging of all the previous components may comprise, consist essentially of, or consist of coating, wrapping, and/or pressing the components into a material such as polydimethylsiloxane (PDMS), polyethylene oxide, plastic, metal, and/or combinations thereof. Alternately, the invention may not include a protective packaging.

The method of fabrication may comprise, consist essentially of, or consist of layer-by-layer assembly, direct electrochemical deposition, electroplating, chemical vapor deposition (CVD), physical vapor deposition (PVD), drop casting, electrospinning, screen printing, inkjet printing, 3D printing, spin coating, and/or combinations thereof.

### EXAMPLES

To fully realize the potential of graphene for implantable capacitive energy storage, bECs were fabricated using novel biophilized graphene oxide (bGO) nanocomposite featuring GO sheets modified with cBSA. The latter material is BSA with chemically attached protonated amine groups to enhance positive charge and proton transfer capacity. LbL deposition was then used to fabricate electrodes of alternate layers of bGO and myoglobin (Mb) onto an 1.0 cm² ultrathin gold sheet to form several graphene oxide modified proteins (bGO/Mb) bilayers (in this example, one bilayer refers to one bGO layer). Then, bGO/Mb was electrochemically reduced to brGO/Mb (brGO is biophilized reduced graphene oxide). A film of three layers of brGO sandwiching two layers of Mb gave the largest capacitance per unit volume, up to 534 F cm⁻³ in human urine at 2.5 A g⁻¹ (contribution of current collector and separator is subtracted). bEC devices made from these electrodes were able to utilize cell culture media and mammalian biofluids as electrolytes for high capacitance output with no measurable signs of cytotoxicity in cell culture media.

In this design, cBSA and Mb should render graphene more compatible with living cells, and provide a good source of heteroatoms to improve the specific capacitance of graphene by introducing pseudocapacitive behavior. These proteins have a good adsorption character and their charge can be easily controlled by changing the pH, which make them an excellent candidate for electrode fabrication. Moreover, proteins act as polyionic nanospacers to prevent the restacking of graphene that often limits the access of the ions to the sheets and undermines its electrochemical performance. A gold current collector was employed in the cell design, since gold is among the safest materials for medical applications. Other designs exist using reduced graphene oxide assembled with polymers that achieved good capacitive performance, but using toxic polymers as part of the electrode materials and/or using toxic electrolytes saturated separators potentially limit these designs' use for implantable biomedical applications.

In order to understand the capacitive behavior of the graphene-protein bECs, different protein films were LbL assembled with brGO (three brGO layers sandwiching two protein layers) and their electrochemical performance was evaluated using charge/discharge curves and cyclic voltammetry (CV). Utilizing calf serum as electrolyte at a current density of 0.005 mA cm⁻², Mb showed the highest areal capacitance reaching 1526 μF cm⁻², followed by cytochrome-C (Cyt-C) with a capacitance of 1175 μF cm⁻², then hemoglobin (Hb), glucose oxidase (GOx), and finally catalase (Cat) in order of decreasing areal capacitance.

Proteins are rechargeable by protonation/deprotonation through their charged residues, such as charged amino acid side chains (lysine, arginine, histidine, glutamic acid, and aspartic acid). Protonation/deprotonation can be used by organic compounds to store the capacitive charge. By calculating the total number of charged amino acid residues (protonatable sites) in each protein and normalizing them to the molar mass of the protein, a strong correlation with the areal capacitance was discovered. Myoglobin had the highest ratio of charged residue/molar mass (Z/M) and the highest areal capacitance, followed by Cyt-C, Hb, and GOx in order of decreasing both the Z/M ratio and the areal capacitance. Catalase showed moderate Z/M ratio but exhibited low areal capacitance, perhaps due to its large molecular size and highly negative hydropathy index. This suggests a key role of the number of protonatable sites in the capacitive performance of the graphene-protein films. Based on these results, Mb nanocomposite with brGO was further explored for the development of bECs. Although myoglobin showed the highest Z/M and areal capacitance, cytochrome-C, hemoglobin, glucose oxidase, and catalase (among others) may also show suitable performance and are explicitly contemplated herein.

Characterization of the brGO/Mb electrode using scanning electron microscopy (SEM) revealed that brGO/Mb films adsorbed to the striated gold current collectors were continuous and relatively uniform. Tapping mode atomic force microscopy (AFM) images showed a uniform first layer of poly(diallyldimethyl ammonium) chloride (PDDA). The adsorption of bGO resulted in a continuous layer of overlapping GO sheets with small islands of cBSA. The incorporation of the Mb layer increased the film roughness, perhaps due to its globular nature. Addition of another layer of bGO on this surface resulted in a sheet-like coating on the Mb layer. Overall, AFM images revealed a homogeneous coating of the LbL film on the gold current collector.

Quartz crystal microbalance (QCM) was used to monitor and control the LbL assembly of the electrode material. The bGO/Mb film was observed to grow almost linearly as a function of the number of layers. The electrochemical reduction of this bGO/Mb film is associated with a decrease in the electrode mass and thickness, suggesting the successful removal of residual oxygen functionalities from the surface of the graphene oxide. QCM estimates a nominal thickness of 32±2 nm for the brGO/Mb active film, while SEM cross section imaging after cutting with a focused ion beam gave 26±5 nm for the same film. Thus, the average thickness of brGO/Mb/brGO/Mb/brGO from the SEM and QCM was 29±4 nm. Analysis of the films by Raman spectroscopy showed an increase in the ratio of the intensities of the D band to the G band (I_{D}/I_{G}) for brGO (1.1) compared to bGO (0.94), suggesting that the reduction resulted in structural modification of graphene oxide sheets with the introduction of defects. Moreover, the intensities of the 2D and S3 bands showed notable increases after reduction, suggesting better graphitization in brGO.

The unique spacing achieved by Mb spreading on the bGO reduced the extent of π-π stacking between adjacent layers and separated them more efficiently. This was confirmed by the shift of the characteristic GO X-ray powder diffraction (XRD) peak in the bGO/Mb film to a lower diffraction angle as compared to the as-prepared GO, indicating an increase of the intersheet distance due to Mb spacing. In addition, Raman spectra of different proteins bound directly to GO were compared to the Raman spectrum of the GO-only sample. Results showed that the Mb-GO film had a sharp, relatively high intensity 2D peak compared to other proteins-bound GO or the as-prepared GO sample, indicating fewer layers of GO sheets and hence better spacing in case of GO/Mb films. This may be attributed to the small size and the higher electrostatic attraction of Mb to GO because of Mb's higher Z/M ratio.

Binding of Mb to bGO was quantified and the bound protein structure examined by circular dichroism (CD) spectroscopy. CD spectra indicated a loss of secondary structure of the bound Mb. Other proteins were also tested as alternatives to Mb; most of these retained their secondary structures on binding to bGO, but capacitive performance was the best with Mb. Proteins in the film increased the capacitance per unit area, up to five times when compared to the capacitances of the corresponding films made from drop-casted reduced graphene oxide, which lacked the proper spacing of sheets and the pseudocapacitance of the heteroatom-rich interlayers with charged residues and protonatable sites, which are required for charge storage by protonation/deprotonation as the brGO layers are charged up. In addition, brGO interlayered with Mb (brGO/Mb) showed two times higher capacitance versus brGO-only films drop-cast on the electrodes, indicating the important capacitive role of Mb in brGO/Mb films.

The electrochemical reduction of the film was achieved in 10×10⁻³ m acetate buffer, pH 5.5 containing 0.5 m potassium bromide (KBr) at constant potential −1.2 V versus saturated calomel electrode (SCE) for 70 s. The reduction of bGO in the film to brGO was indicated by the change of film color from brown to black, and is supported by a reduction peak for bGO found at −0.95 V versus SCE by CV that disappears upon subsequent scans. In addition, a dramatic increase in capacitance was found after bGO reduction to brGO. CV profiles showed characteristic Fe^{III}/Fe^{II} peaks of Mb at −0.3 V versus SCE in acetate buffer. These characteristic peaks appeared before and after reducing the film for 3 s, but continued reduction for 70 s caused a large increase in the charging current and loss of the Mb peaks.

The iron heme center of heme proteins is an important active center for a number of catalytic reactions, and was also reported to contribute to capacitance of thick proteins films supported on nickel foam electrodes. However, loss of the characteristic redox peaks of the Fe^{III}/Fe^{II} of the heme center after electrochemical reduction, along with the loss of the protein secondary structure as revealed by CD, confirm that Mb is denatured in the brGO/Mb electrodes and the heme center is lost or deactivated, which suggests a minor role of the Fe^{III}/Fe^{II} heme redox couple in the capacitive behavior of the brGO/Mb electrodes.

Myoglobin has the highest Z/M ratio among the studied proteins, and yet Mb-only films had low specific capacitance of 16.6 F g⁻¹. The low capacitance of Mb in the absence of graphene is perhaps due to the poor electrical conductivity of Mb-only films. The electrical conductivity of the Mb alone and in the LbL assembled film with brGO were measured using the 4 probe technique. Mb showed low electrical conductivity of 3.6×10⁻⁸ cm⁻¹ at room temperature due to its insulating nature, which perhaps explains the poor electron flow through thick Mb films and its low specific capacitance. However, the introduction of brGO to the Mb films increased the electrical conductivity by five orders of magnitude reaching 0.003 S cm⁻¹ at room temperature. The electrical conductivity of the brGO/Mb electrode was further explored at different temperatures using a two-electrode setup. The brGO/Mb electrode showed good thermal stability and increase in conductivity as the brGO/Mb electrode was heated up to 150° C., suggesting that the brGO/Mb electrodes are thermally stable at temperatures far beyond those needed for implantable applications.

The electrochemical performance of the brGO/Mb electrodes was further studied to assess capacitive efficiency as implantable bECs. The capacitive behavior depended strongly on the number of bilayers. The areal capacitance increased almost linearly up to three bilayers, after which a plateau was observed. This is likely due to the low conductivity of the Mb layers that limit electron transport between the top layer and the current collector for thicker films. The capacitance functionality of each of the five layers of the brGO/Mb film and the supports were evaluated in calf serum using charge/discharge curves at 0.005 mA cm⁻². The areal capacitance of the positively charged gold current collector (with PDDA adsorbed on the surface) was 90 g cm⁻², which represents 6% of the areal capacitance of the final brGO/Mb electrode. The capacitance of the current collector was subtracted from the capacitance of all other samples. The adsorption of the first bGO layer followed by electrochemical reduction to brGO resulted in 2.0 μg cm⁻² brGO layer, as measured by QCM, with an areal capacitance of 462 μF cm⁻² and a gravimetric capacitance of 232 F g⁻¹. The first brGO layer represents 40% of the mass of the final brGO/Mb film and 30.3% of its areal capacitance. The adsorption of the positively charged Mb as a second layer resulted in a significant increase in the areal capacitance, reaching 847 μF cm⁻² and increasing the gravimetric capacitance of the assembled film to 260 F g⁻¹, indicating the key role of myoglobin in enhancing the capacitive performance of the device. The areal capacitance increased by adding more brGO and Mb layers, achieving the maximum areal and gravimetric capacitance after five layers (brGO-Mb-brGO-Mb-brGO) with 1526 μF cm⁻² and 306 F g⁻¹, respectively. A similar film was prepared by replacing the bGO with GO for the LbL assembly with Mb followed by electrochemical reduction to form rGO-Mb-rGO-Mb-rGO electrode, abbreviated as (rGO/Mb), resulting in a gravimetric capacitance of 239 F g⁻¹, which is 22% less than the brGO/Mb electrode. The significant capacitance contribution of cBSA is possibly due to its high charged amino acid content with 198 protonatable sites per molecule, scoring the second highest Z/M ratio (0.003) of the studied proteins after Mb. These results show the capacitive contribution of the proteins (cBSA and Mb) in enhancing overall capacitive behavior. Based on these results, electrodes of three brGO layers alternated with two Mb layers achieved the maximum capacitance and were used for subsequent device fabrication.

A prototype implantable bEC was assembled from two brGO/Mb/brGO/Mb/brGO electrodes bound to a flexible thin gold foil with a thickness of 42±8 nm, obtained after etching away a plastic protective layer of a gold CD. To get a neat and ultrathin EC, a layer of polyvinyl acetate gel saturated with electrolyte was loaded onto the electrodes to serve as both the separator and electrolyte with an average separator thickness of 992 nm. This simplified the processing of the electrodes into paper-like flexible bECs. The total thickness of the device was 1.14 μm, providing a thin, lightweight power source for implantable devices.

The packageless brGO/Mb bEC effectively utilized different biofluids in the surrounding medium as electrolytes, including calf serum, cell culture media, and human urine. For comparison, the brGO/Mb bEC in 0.1 m sulfuric acid, a typical standard electrolyte, was also tested. It is important for ECs with high energy density to be characterized at high scan rates, especially when used as a power source for implantable bioelectronics. This enables the fast charging of the current received from the nanogenerator and the fast discharging of the electrical signal to the implantable devices by the bEC. Therefore, the bEC device capacitive performance at moderate-to-high current density and high scan rate ranges was presented. Results show nearly rectangular CV shapes even at high scan rates of 1000 mV s⁻¹, indicating nearly ideal capacitive behavior. The maximum volumetric capacitance in biofluids (after subtracting the contribution of current collectors) was 655 F cm⁻³ at scan rate of 100 mV s⁻¹ and 534 F cm⁻³ obtained at a current density of 2.5 A g⁻¹ in human urine, while the volumetric capacitance in calf serum was 563 F cm⁻³ at 100 mV s⁻¹ and 372 F cm⁻³ at 2.5 A g⁻¹. The capacitance may depend on the availability and type of ions present in these different electrolytes, illustrated by a 14% drop in capacitance when changing from human urine to calf serum. The short-term cycling stability of the packageless bEC turns out to be excellent with only a 2% drop in capacitance after 5000 cycles in calf serum. This good cycling performance in biofluids suggests the short-term stability of the packageless bECs operating in biological environment, while the long-term stability and toxicity of the device will be further tested when the device is dipped in living cell culture.

The excellent performance of brGO/Mb bECs was also confirmed from charge/discharge cycles. Depending on the type of electrolyte, the brGO/Mb bEC can provide a high stack capacitance of 13 to 16 F cm⁻³. This was calculated based on the volume of the device stack, which includes the gold current collector, active materials, electrolyte, and separator. When compared with commercial electric double layer capacitors (EDLCs), the brGO/Mb can provide 25 to 400 times higher stack capacitance. Not only does the brGO/Mb device provide higher capacitance, but it also exhibits excellent rate capability at high current densities up to 20 A cm⁻³. This remarkable performance may be attributed to the microstructure of the electrode featuring brGO/Mb layered films with different pore sizes due to the protein interlayers, which may facilitate the ion movement during charge/discharge process.

In order to demonstrate the overall performance of the brGO/Mb bEC, its energy density and power density in a Ragone plot were calculated. The brGO/Mb bEC displays a high energy density of 1.1 W h kg⁻¹ at a high power density of 116 W kg⁻¹, calculated based on the total mass of the device stack. The same plot was produced using the volume of the device stack. For comparison, the energy density and power density were tested of a 300 μF/3 V aluminum electrolytic capacitor, a 12 μA h/3.3 V Li thin film battery, a 500 μA h/5 V Li thin film battery, and three commercially available EDLCs designed for small-scale applications. The brGO/Mb bEC can provide a high energy density of 1.8 mW h cm⁻³, which is comparable to Li thin film batteries and 3 to 11 times higher than thin film commercial EDLCs. The brGO/Mb device can also deliver 100 times higher power density than Li thin film batteries, which is sufficient to drive all known implantable medical devices. It is worth mentioning that a purpose of this design is for ultrathin bECs for miniaturized implantable devices and therefore a comparison with conventional thick film electrodes is irrelevant for these applications.

The brGO/Mb system described herein is the first graphene-protein bEC, and shows superlative characteristics as a power source for implantable devices. For example, proteins have been used as a source for carbon electrodes by graphitization at 800° C. These devices do not feature intact proteins and have low capacitance. Redox hemin-based proteins were used to enhance pseudocapacitance on a nickel foam, but again low specific capacitances were obtained (e.g., hemoglobin 12 F g⁻¹¹), possibly due to the low conductivity of the protein-only films. In addition, supercapacitors fabricated from a biofilm of the conductive bacteria Geobacter sulfurreducens, which is rich in cytochrome C protein, was reported. Using the live conductive bacterial biofilms as electrode material resulted in low specific capacitance, potential loss of conductivity after the bacteria die, and risks of infection if used in implantable biomedical devices. On the other hand, previous reports of DNA based ECs have been tested for their adverse effects on living cells, and showed moderate-to-no toxicity signs on cells, but they had at best six times lower gravimetric capacitance than brGO/Mb bECs.

Toward developing safe implantable bECs, toxicity and adverse effects of materials used to fabricate the bEC electrodes were studied using living mammalian cells. The toxicity of different concentrations of the as-prepared GO and bGO/Mb on COS-7 and mouse embryo fibroblast (MEF) cell lines was evaluated by optical microscopy and spectrophotometrically by employing WST-8 dye (water-soluble tetrazolium salt) as a chromogen. WST-8 is a water-soluble, cell-permeable dye, which gets reduced by the active intracellular dehydrogenases (IDH) in the cell cytosol. Quantitative reduction of WST-8 by IDH enables accurate quantification of the overall metabolic health of a living cell.

The native GO showed significant cell damage after being co-incubated with COS-7 cells for 8 h at a 25 μg mL⁻¹ dose, resulting in 41% specific cell death as measured by trypan blue assay, significant cell shrinkage, and cell cycle arrest, and 60% drop in IDH activity. In contrast, no sign of cell toxicity was observed for bGO/Mb co-incubated with COS-7 cells for 48 h at a dose as high as 1600 μg mL⁻¹ with no statistically significant change in live/dead cell ratio or IDH activity compared to control cells. These results suggest that native GO may cause serious toxic effects and the metabolic health of live cells and perhaps should not be used directly in contact with human cells. On the other hand, bGO/Mb was compatible with cells even at very high concentrations with little change in toxicity or metabolic health of cells at doses 160 times higher than those used in the bECs. Results suggest that bGO/Mb is safe for living cells, and can be used for fabricating implantable packageless bECs working in open cell solution or encapsulated bECs.

Performance and toxicity of the packageless brGO/Mb bECs were evaluated in cell cultures of MEF and COS-7 cells in Dulbecco's modified Eagle medium (DMEM) while these bECs were subject to continuous cycling test. brGO/Mb bECs were placed in the culture medium with cells previously grown in six-well plates. The packageless bEC was capable of utilizing ions in the cell culture medium for charging and discharging. CVs at different scan rates showed nearly rectangular shapes up to 500 mV s⁻¹ indicating almost ideal capacitive behavior. The toxicity of the brGO/Mb bEC was tested by charging and discharging the packageless device in a MEF and COS-7 cell medium for 5000 cycles at high current density for 3 h. No change in cell morphology or dead/live cell ratio was found due to the cycling test, indicating no toxic effects on live cells on short term as measured by the trypan blue assay. The cell viability after the charge-discharge regimen was found to be identical and no significant differences with respect to the control cell population (p<0.01, paired student's t-test).

The brGO/Mb in a packageless platform makes the bEC devices ultrathin, light weight, and completely flexible. However, before using the packageless platform of the bEC device in any long-term implantable applications, the stability of the device components has to be evaluated for long-term use. The long-term stability of the brGO/Mb electrode was evaluated using QCM after the film was exposed to 96 h of continuous charge and discharge cycles in water (50×10⁻³ M NaCl was added to provide ions for the charge-discharge process) in open cell solution. Results showed a small decrease in the mass of the film from 5.0 to 4.7 μg cm⁻², which indicates a stable brGO/Mb electrode. However, the polyvinyl acetate (PVA) separator of the packageless sandwich device showed limited stability in open cell solution, and started to break and diffuse into the surrounding solution after only 27 h. Thus, polyvinyl acetate may not be a suitable separator for the long-term cycling of the packageless bECs in open cell solution.

Encapsulating the brGO/Mb device in polydimethylsiloxane (PDMS) was evaluated. The PDMS was chosen as a packaging material because of its low toxicity, flexibility, and light weight. bECs showed the same electrochemical behavior with and without PDMS packaging while maintaining complete flexibility. Moreover, the PVA separator remained intact throughout the long-term cycling of the PDMS-packaged bECs for four continuous days (90,192 cycles) while the bEC was immersed in COS-7 cell culture medium. Cycling for four days provided enough time for the cells to make two full cycles of cell division. During the entire charging-discharging process, the growing cells were monitored by optical microscopy and no perceptible difference in the cell multiplication and overall morphology was found between control and bEC test cells. No perceptible difference in the overall IDH metabolic activity was found between control cells and the cells subjected to continuous charging and discharging for 96 h. Results show that PDMS device packaging is efficient in maintaining the integrity of performance with no toxic effects on cells. In addition, the drop in the areal capacitance after cycling the device for 96 h was only 11%. These results suggest that brGO/Mb devices could work efficiently as implantable power sources in packageless platform for short term and for long term in PDMS-encapsulated design with good stability.

Since implantable bioelectronics are always subject to movement inside the patient's body, brGO/Mb bEC performance was evaluated at different bending conditions. The ultrathin brGO/Mb bEC showed excellent tolerance for bending to different angles with no change in capacitance even after bending to 180°. However, a slight decrease in capacitance was observed when the device was subjected to 1000 charge/discharge cycles while bent at 90°. In addition, the brGO/Mb device showed no loss of performance after bending to 1,000 bending cycles to an angle of 90°. These results suggest that the brGO/Mb bECs are robust under bending stresses in both packaged and packageless designs, which make these bEC suitable for the different bending conditions expected for implantable bioelectronics.

Electrochemical impedance spectroscopy showed nearly straight lines in the high-frequency region in all tested electrolytes. The brGO/Mb device showed multiple time constants behavior with cell time constant of only 1.5 ms in biofluids at −45°. In addition, the down-turn of the phase angle at low frequency (less than 1 Hz) is mainly due to the leakage current/voltage from the packageless devices. Three packageless bECs made up of the ultrathin brGO/Mb films were yet capable of efficiently powering a light emitting diode utilizing calf serum as the electrolyte. On the other hand, the rapid performance of the devices may be related to fast ion movement through thin electrode assemblies, with myoglobin possibly acting as a nanochannel for fast ion transport.

Novel brGO-protein bECs were fabricated with high volumetric capacitances up to 655 F cm⁻³ in biofluids. Proteins bound to GO sheets played multiple roles in fabricating safe and high-performance bECs. First, proteins rendered the GO sheets nontoxic to cell-containing environments, even at high concentrations. Moreover, proteins provided pseudocapacitive behavior for charge/discharge through their heteroatom-rich nanostructures and the high abundance of the protonatable charged amino acid residues. Finally, protein layers provided nanopores that may act as nanochannels for shuttling ions in the biofluids to the conductive graphene interlayers. These devices retained high energy density comparable to that of Li thin film batteries while utilizing serum/urine or cell media as electrolytes to power LEDs with little loss of performance, and no cell toxicity after 5000 cycles in packageless platform and 90,000 cycles in PDMS-packaged design. Unlike batteries that suffer from relatively slow discharge rates, the ultrathin brGO/Mb bECs have much faster performance, making these devices good for the fast discharge of electrical signals to implanted biomedical devices. With protein as a major component and utilizing body fluids for electrolyte function, these devices have the potential to power the next-generation miniaturized bioelectronics.

### DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrase “at least one of A, B, . . . and N” or “at least one of A, B, . . . N, or combinations thereof” are defined in the broadest sense to mean one or more elements selected from the group comprising A, B, . . . and N, that is to say, any combination of one or more elements A, B, . . . or N including any one element alone or in combination with one or more of the other elements, which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4”. The term “about” may refer to plus or minus 10% of the indicated number. For example, “about 10%” may indicate a range of 9% to 11%, and “about 1%” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

Thus, the invention provides, among other things, a supercapacitor including reduced biophilized graphene oxide and a protein nanospacer. Various features and advantages of the invention are set forth in the following claims.

What is claimed is:

1. A supercapacitor comprising bilayers of reduced biophilized graphene oxide and a protein nanospacer.

2. The supercapacitor of claim 1, wherein the reduced biophilized graphene oxide comprises a cationized protein.

3. The supercapacitor of claim 1, wherein the protein nanospacer comprises myoglobin.

4. The supercapacitor of claim 1, wherein the number of bilayers is three, such that there are two individual layers of protein nanospacer and three individual layers of reduced biophilized graphene oxide.

5. The supercapacitor of claim 1 further comprising a current collector.

6. The supercapacitor of claim 5, wherein the current collector comprises a gold sheet about 34 nm to about 50 nm thick.

7. The supercapacitor of claim 1 further comprising an electrolyte, wherein the electrolyte comprises a biofluid.

8. The supercapacitor of claim 1, wherein the supercapacitor shows essentially no cell toxicity at operating concentrations.

9. The supercapacitor of claim 1, wherein the supercapacitor is less than or equal to about 1.14 μm thick.

10. The supercapacitor of claim 1, wherein the supercapacitor exhibits a capacitance of at least about 650 F/cm³ at a scan rate of about 100 mV/s.

11. The supercapacitor of claim 1, wherein the supercapacitor exhibits a capacitance of at least about 530 F/cm³ at a current density of about 2.5 A/g.

12. The supercapacitor of claim 1, wherein the supercapacitor exhibits a capacitance of at least about 860 F/cm³ at about 25 mV/s.

13. The supercapacitor of claim 1, wherein the supercapacitor exhibits a current density of at least about 1.8 mWh/cm³.

14. The supercapacitor of claim 1, wherein the supercapacitor exhibits an energy density of at least about 1.1 mWh/g.

15. The supercapacitor of claim 1, wherein the supercapacitor exhibits a power density of at least about 13.5 W/g.

16. The supercapacitor of claim 1, wherein the supercapacitor is capable of delivering a power density of at least about 5 mW/cm³.

17. The supercapacitor of claim 1, wherein the supercapacitor is capable of a frequency response characterized by a cell time constant of less than or equal to about 1.5 milliseconds.

18. A method of producing the supercapacitor of claim 1, the method comprising:
(a) Mixing bovine serum albumin and tetraethylenepentamine into a mixture;
(b) Adding a crosslinking agent to the mixture;
(c) Adding the mixture to graphene oxide;
(d) Providing a current collector having a surface;
(e) Adsorbing poly(diallyldimethyl ammonium) chloride to the surface of the current collector;
(f) Adsorbing a layer of the graphene oxide mixture directly or indirectly to the surface of the current collector;
(g) Adsorbing a protein nanospacer directly or indirectly to the surface of the current collector;
(h) Optionally repeating steps (f) and (g); and
(i) Reducing a film comprising the graphene oxide mixture layers in step (f) and the protein nanospacers in step (g).

19. An implantable biomedical device comprising the supercapacitor of claim 1.

20. The implantable biomedical device of claim 19, wherein the implantable biomedical device is a pacemaker.

21. The implantable biomedical device of claim 19, wherein the supercapacitor is capable of powering the implantable biomedical device for the lifetime of a patient.

22. The implantable biomedical device of claim 19 further comprising a standalone power source.

23. The implantable biomedical device of claim 22, wherein the standalone power source comprises a biosupercapacitor coupled with an energy harvester.

24. The implantable biomedical device of claim 23, wherein the energy harvester comprises a triboelectric nanogenerator (TENG), a piezoelectric energy harvester, or a thermoelectric energy harvester.

25. A portable electronic device powered by the implantable biomedical device of claim 23.

26. The portable electronic device of claim 25, wherein the portable electronic device is a cell phone, a laptop computer, or a tablet.

27. The supercapacitor of claim 1, wherein the supercapacitor has essentially no cell toxicity at a dose of about 1600 μg/mL.

28. The supercapacitor of claim 1, wherein the supercapacitor can be charged through wireless charging to power portable biosensors, implantable deep brain stimulator, or cardiac pacemaker.

29. A portable biosensor, an implantable deep brain simulator, or a cardiac pacemaker powered by the supercapacitor of claim 28.
